(19) United States
(12) Patent Application Publication
(10) Pub. No.: US 2018/0153206 A1
(43) Pub. Date: Jun. 7, 2018

(54) Labile-Based Spray and Emissions for Food Applications

(71) Applicant: Kamterter Products, LLC, Waverly, NE (US)

(72) Inventors: John Alvin Eastin, Waverly, NE (US); David Vu, Waverly, NE (US)

(21) Appl. No.: 15/807,318

(22) Filed: Nov. 8, 2017

Related U.S. Application Data
(60) Provisional application No. 62/419,205, filed on Nov. 8, 2016.

(51) Int. Cl. A23P 20/18; G01N 11/02; G01N 33/02

(52) U.S. Cl. CPC A23P 20/18; G01N 33/02; G01N 11/02

(57) ABSTRACT

Food applications including seed treatment, planting, pest control, plant health, harvest and desiccation, processing, preservation, flavor enhancement, encapsulation, suspension, emulsion techniques, packaging, shipping, and storage applications incorporate low pressure or low energy processes to emit viscous fluids. Fixtures, applicators, application configurations, and operational parameters and dimensions may be determined, limited, and selected based on a lability characteristic of a feedstock ingredient with respect to a lability reference frame.

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)). All subject matter of the Related Applications and of any and all parent, grandparent, great-grandparent, etc. applications of the Related Applications is incorporated herein by reference to the extent such subject matter is not inconsistent herewith.

### RELATED APPLICATIONS

The present application is related to U.S. Patent No. 62/419,205, entitled SYSTEMS FOR THE CONTROL AND USE OF FLUIDS AND PARTICLES IN FOOD APPLICATIONS INCLUDING SEED TREATMENT, PLANTING, PEST CONTROL, PLANT HEALTH, HARVEST AND DESICCATION, PROCESSING, PRESERVATION, FLAVOR ENHANCEMENT, ENCAPSULATION, SUSPENSION, EMULSION TECHNIQUES, PACKAGING, SHIPPING, AND STORAGE, naming John Alvin Eastin and David Vu as inventors, filed Nov. 8, 2016.

### BACKGROUND

It is known to shape and spray fluids and suspensions with spraying systems. In some applications, the fluids and suspensions are formed into droplets or aerosols and sprayed by the spraying systems. In other applications, the fluids form particles or capsules about seeds or chemicals.

One use of such spraying systems is to apply agricultural inputs to seeds for a seed treatment application. Commonly, the spraying systems include applicators with pumps that carry the pressurized agricultural inputs to spray equipment that apply the agricultural inputs from nozzles supported by booms on the applicator. The pumps may be utilized for distribution of the agricultural inputs.

In many food applications, exposure of ingredients to reactants may unintentionally convert the ingredients to unwanted products, or may result conversion before an intended point in time. High levels of viscosity may make delivery impractical or less cost effective for conventional systems. Pressurized conventional systems or increasingly turbulent flow may damage sensitive biologicals, reducing or eliminating efficacy. Hydrophilic solutions are often unstable or subject to alteration without additional additives.

The prior art spray systems have several disadvantages such as for example: (1) they require vehicles carrying agricultural inputs or metabolic inputs at weights that are higher than desirable weights due to the associated water carrier; (2) they require the replenishment of the supply of agricultural inputs carried by the spray vehicles periodically, thus increasing the time and expense of spraying; (3) they cannot be used for the application of some beneficial microorganisms because the microorganisms are killed by the high pressure drop experienced by the microorganisms upon release through the spray nozzles used in the prior art techniques for application of agricultural inputs; (4) the low viscosity agricultural inputs or metabolic inputs drift or evaporate when sprayed as small drop sizes; (5) some of the carriers used for dilution, such as water, have high surface tension and form beads on contact rather than spreading such as over a leaf or a surface of an edible seed; (6) the low viscosity sprayed drops tend to break up because of low shear resistance, thus forming smaller drops that are subject to increased drift; (7) some of the carriers used for dilution, such as water, have unpredictable mineral content and pH variations; (8) the angle of the pattern of sprayed fluid from the nozzles is limited thus requiring the nozzle to be positioned at a high elevation above the spray target to obtain adequate coverage but the high elevation increases drift; (9) the use of some combinations of active ingredients in conventional carriers in some circumstances causes precipitation of active ingredients (10) the prior art systems cannot effectively spray some particles such as particles that have absorbed active ingredients in them that are to be released at a later time and/or environmental condition or over a timed interval because for example they cannot spray viscous formulations that facilitate suspension of such materials; (11) the angle over which the spray is released for hydraulic nozzles is less flexible in prior art nozzles resulting in target coverage limitations; (12) the conventional high pressure hydraulic atomization nozzles used result in excessive nozzle wear and consequential variations in the distribution rate and frequent changes in nozzles; (13) sprayer vehicle speed is limited by the pressure because higher pressures are required for high rates of application and that results in small droplets that drift and there are pressure limitations on the system components; and (14) some of the materials used for carriers are low density and/or evaporate quickly thus increasing the tendency to drift. Moreover, in some instances, the drops lose some carrier by evaporation and the drops end up with concentrations of materials that cause necrosis of plants that are not intended to be adversely affected by the spray.

The prior art attempts to reduce drift have been faced by a dilemma—small drop sizes increase drift problems but provide good coverage of the target and large drop sizes reduce drift but provide poor coverage of the target. The higher concentration sprays have an increased tendency to cause necrossis of plants. For example, some compositions of glyphosate sprays concentrate on plants immune to the effects of glyphosate (Round-up Ready crops are engineered to resist the toxic effects of glyphosate. Round-Up Ready is a trademark of Monsanto Company.) The prior art attempts to resolve this dilemma by compromising between drop size and drift and selecting special nozzles. The special nozzles rely upon air injection into the liquid to facilitate atomization and assist in reducing liquid pressure or the use of liquid pulse modulation systems. These prior art approaches have disadvantages of still providing coverage less effective than desired or longer drift distances than desired and are limited in the application rate adjustments that can be practically achieved as application vehicles change speed in order to maintain constant per unit of field area application rates.

Higher density materials have been available to use as carriers for active ingredients but have not been used because of economic reasons or undesirable characteristics or the belief that such materials would be difficult to spray because of their viscosity or density or because of the custom of using water as the primary carrier material. Many of the active ingredients are difficult to spray with prior art stand-alone nozzles or air assist nozzles because they principally require pressure against an orifice to meter and atomize the materials and that cannot be reasonably accomplished with viscous liquids. The long established and reasonable practice has been to dilute the high viscosity active ingredients with low viscosity mobile carriers such as water. However, it has been found that this general prior art approach is not the best approach and has the disadvantage of resulting in a low concentration, higher weight and higher volume load carried by the spray vehicle than is desirable.

It is known to mix fluids and particles and to chemically or physically interact them. Some coating processes, for example, physically interact materials to encapsulate one within the other. There are many such procedures that shape, mix and interact different fluid materials for useful purposes.

The prior art processes for mixing fluids and particles and chemically or physically interacting them have some common disadvantages. For example, the size of particles or drops or phase of the materials being mixed may not be as appropriate as possible, the selection of materials or proportion of different materials to be interacted may lack some materials or include too many materials or not have a sufficient quantity of some materials or the timing of the interacting of materials may not be suitable or the material compatability may be for example time or concentration sensitive.

It is also known to use screw type mechanisms that receive and capture seeds carried along by a fluid such as air or water and emit the seeds one by one. Such an apparatus is disclosed in U.S. Pat. No. 2,737,314 to Anderson. This apparatus has a disadvantage of damaging seeds and being relatively complicated and unreliable. Augers are known for conveying matter from place to place but such augers have not been successfully adapted up to now to fluid drilling apparatuses. Some such augers have utilized a stream of air at an angle to the flow of material to break off controlled lengths of the material and such an apparatus is disclosed in U.S. Pat. No. 3,846,529. However, this patent does not disclose any method of fluid drilling. The augers used in the prior art are not designed in a manner adequate to separate seeds, to avoid plugging of the conduits carrying the seeds and gel to the nozzle from which they are to be expelled into the ground nor to maintain spacing between seeds while moving them along the auger.

### SUMMARY

This invention relates to the forming, shaping, control and use of fluids and particles in food applications such as for example in seed treatment, planting, pest control, plant health, harvest and desiccation, processing, preservation, flavor enhancement, encapsulation, suspension, emulsion techniques, packaging, shipping, and storage. One example is the formulation of metabolic inputs, shaping them into droplets or particles, and the distribution of the droplets or particles over a seed for seed treatment. Another example is for the encapsulation of seeds or biologicals with or without chemicals and other biological agents or the encapsulation of chemicals with or without biological agents and distribution of the encapsulated seeds or chemicals or biological materials. Still another is for the suspension of seeds for fluid drilling of seeds with or without chemicals or biological materials.

It is an object of the invention to provide a novel apparatus for spraying viscous materials.

It is a still further object of the invention to provide a novel method for spraying viscous materials.

It is a still further object of the invention to provide a novel method for applying large numbers of discrete portions of a material to surfaces such as for example applying material to leaves or to a field with increased efficiency.

It is a still further object of the invention to provide a novel method and apparatus for encapsulating materials.

It is a still further object to mix labile ingredients with precision to avoid degradation. It is a still further object to mix immiscible liquids with precision for improved uniformity in droplet concentration or droplet application.

It is a still further object to mix high viscosity fluids, or have suspended therein, solid particles to deliver suspensions and/or emulsions for a desired food application. It is a still further object to deliver these suspensions and/or emulsions according to lability characteristics including physical and energy characteristics resembling those of non-Newtonian fluids.

It is a still further object to limit applicator selection criteria based on lability characteristics of a desired feedstock.

It is a still further object to limit configuration criteria based on lability characteristics of the desired feedstock.

Methods and apparatuses related to fluid drilling, seed mixing, spray fixtures, nozzles, delivery mechanisms, distribution mechanisms, distributing chitosan, planting, apparatuses for planting, mixing and/or distributing immiscible ingredients, and combinations thereof, are described generally in U.S. Pat. No. 9,148,994, issued on Oct. 06, 2015, filed Nov. 12, 2012, by John Alvin Eastin, et al., titled SYSTEMS FOR THE CONTROL AND USE OF FLUIDS AND PARTICLES, which is incorporated herein by reference in its entirety.

In one aspect, the invention may include a method for labile-based spray and emission for food applications. The method may include an act of determining a lability reference frame for a feedstock. The method may include an act of comparing first lability characteristics of the feedstock at a first point in the lability reference frame with second lability characteristics of the feedstock at a second point in the lability reference frame to determine a level of lability of the feedstock. The method may include an act of limiting a spectrum of selection criteria or configuration criteria based on the level of lability. The method may include an act of converging on a value to determine an adjustment, a calibration, a dimensional, or an operational parameter using the limit and at least one of the first lability characteristics and the second lability characteristics.

In a further aspect, the invention may include a method of spray or emission for food applications based on lability characteristics. The method may include an act of selecting one or more spray or emission applications from among multiple food applications. The method may include an act of determining one or more spray or emission forms and associated spray or emission parameters. The method may include an act of determining feedstock characteristics including one or more of lability, thermodynamic, fluidic, and kinematic properties and associating the feedstock characteristics with a lability reference frame. The method may include an act of providing spray fixture selection or configuration criteria based on the lability reference frame, the feedstock characteristics, the emission form, and the selected spray or emission form.

In a further aspect, the invention may include a system for spray or emission for food applications based on lability characteristics. The system may include an applicator having an adjustable speed. The applicator of the system may include a first port, a second port, and one or more adjustable valves. In this regard, each of the first port and the second port may be configured for interchangeably receiving one or more components based on lability characteristics of a feedstock. The system may include a selected feedstock hopper having a third port configured to interface with the first port of the applicator. The system may include a selected distribution mechanism having a fourth port configured to interface with the second port of the applicator. The distribution mechanism may be coupled with the feedstock hopper via one or more delivery mechanisms and configured to spray or emit the feedstock at least proportionally to the adjustable speed and according to the lability characteristics of the feedstock. In this regard, the spray or emission may be adjustable at least with respect to spray and emission form using the adjustable valve.

### BRIEF DESCRIPTION OF THE DRAWINGS

Implementations of the inventive concepts disclosed herein may be better understood when consideration is given to the following detailed description thereof. Such description makes reference to the included drawings, which are not necessarily to scale, and in which some features may be exaggerated and some features may be omitted or may be represented schematically in the interest of clarity. Like reference numerals in the drawings may represent and refer to the same or similar element, feature, or function. In the drawings:

FIG. 1 is a Venn diagram for limiting selection or configuration criteria, according to the inventive concepts disclosed herein;

FIG. 2 is a flow diagram for a method of labile-based spray and emission for food applications;

FIG. 3 is a flow diagram of a method for spray or emission for food applications based on lability characteristics;

FIG. 3A is a flow diagram of a method for spray or emission for food applications based on lability characteristics;

FIG. 3B is a flow diagram of a method for spray or emission for food applications based on lability characteristics;

FIG. 4 is a flow diagram of a method for spray or emission for food applications based on lability characteristics; and

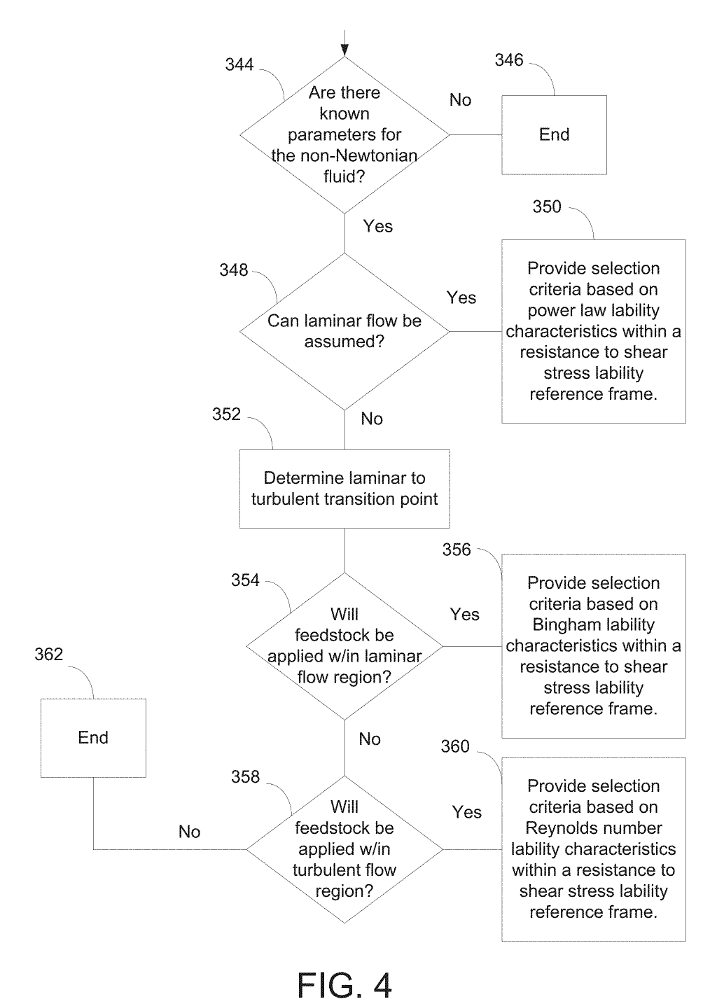

FIG. 5 is a system for spray or emission for food applications based on lability characteristics.

### DETAILED DESCRIPTION OF EXEMPLARY EMBODIMENTS

Before explaining at least one embodiment of the inventive concepts disclosed herein in detail, it is to be understood that the inventive concepts are not limited in their application to the details of construction and the arrangement of the components or steps or methodologies set forth in the following description or illustrated in the drawings. In the following detailed description of embodiments of the instant inventive concepts, numerous specific details are set forth in order to provide a more thorough understanding of the inventive concepts. However, it will be apparent to one of ordinary skill in the art having the benefit of the instant disclosure that the inventive concepts disclosed herein may be practiced without these specific details. In other instances, well-known features may not be described in detail to avoid unnecessarily complicating the instant disclosure. The inventive concepts disclosed herein are capable of other embodiments or of being practiced or carried out in various ways. Also, it is to be understood that the phraseology and terminology employed herein is for the purpose of description and should not be regarded as limiting.

As used herein a letter following a reference numeral is intended to reference an embodiment of the feature or element that may be similar, but not necessarily identical, to a previously described element or feature bearing the same reference numeral (e.g., 1, 1a, 1b). Such shorthand notations are used for purposes of convenience only, and should not be construed to limit the inventive concepts disclosed herein in any way unless expressly stated to the contrary.

Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, use of the “a” or “an” are employed to describe elements and components of embodiments of the instant inventive concepts. This is done merely for convenience and to give a general sense of the inventive concepts, and “a” and “an” are intended to include one or at least one and the singular also includes the plural unless it is obvious that it is meant otherwise.

Finally, as used herein any reference to “one embodiment,” or “some embodiments” means that a particular element, feature, structure, or characteristic described in connection with the embodiment is included in at least one embodiment of the inventive concepts disclosed herein. The appearances of the phrase “in some embodiments” in various places in the specification are not necessarily all referring to the same embodiment, and embodiments of the inventive concepts disclosed may include one or more of the features expressly described or inherently present herein, or any combination of sub-combination of two or more such features, along with any other features which may not necessarily be expressly described or inherently present in the instant disclosure.

“Labile” as used herein includes a broad definition, such as the level at which a thing (e.g., object, molecule, intermolecular interaction, ingredient, reactant, product, or combination thereof) is subject to change or alteration. The level of lability may only be determined by first determining a labile reference frame.

“Emission” or “emit” as used herein shall mean a discharge or to discharge, project, or release.

“Delivery mechanism” as used herein includes, but is not limited to, an auger within a delivery tube, multiple augers within a delivery chamber or a large delivery tube, a low pressure pump (e.g., peristaltic, gear, etc.), inlets and/or outlets, a fluid line, a valve, a hopper sized or angled for effective delivery, an electric charge, a needle-like column, adjustable opposing plates, a spray fixture, capillary forces, and combinations thereof.

“Distribution mechanism” as used herein includes, but is not limited to, a spray fixture, a nozzle, spray outlets, delivery tube outlets, a shearing knife, an opening between a plate of a fixture, and combinations thereof.

“Propellant” as used herein includes a low to medium viscosity fluid used to propel an ingredient, fluid, particle, semi-solid, slurry, emulsion, colloidal suspension, or a combination thereof, out of a distribution mechanism. In some embodiments, the propellant includes only low viscosity fluids. In other embodiments, a medium viscosity fluid may be used as the propellant, depending on characteristics of the feedstock material. The propellant generally combines its kinetic energy with the kinetic energy of the material being propelled (e.g., utilizes mostly constructive forces as opposed to destructive forces). The propellant generally has a lower viscosity than the material it propels. However, there are embodiments in which propelled ingredients are vaporized and/or atomized prior to being delivered, such that in those embodiments, the propellant may have a higher or similar viscosity as compared to the material being propelled.

“Feedstock material” or “feedstock” as used herein includes any ingredients, fluids, particles, semi-solids, slurries, emulsions, colloidal suspensions, or combinations thereof, delivered via a delivery mechanism disclosed herein to a distribution mechanism. The feedstock material generally has a high viscosity, such as a non-Newtonian fluid. The feedstock generally has a higher viscosity than the propellant. However, it is noted that in some applications, feedstock ingredients may be vaporized prior to mixing, and in such cases, the feedstock may have a lower or similar viscosity as compared to the propellant.

“Distributing” as used herein includes any form of moving, collecting, spraying or otherwise disposing of groups, patterns, or individual distributed forms of at least one of the following: fluid flow, drop, slurry, globule, fiber, particle, vapor, or mist.

“Spray fixture” or “nozzle” as used herein includes an apparatus adapted to be connected to a source of feedstock material and to a force for powering the feedstock material through the apparatus, the apparatus including an outlet and structure for controlling the output of feedstock material from the outlet of the spray fixture. The spray fixture encompasses more structure than a nozzle, and therefore in embodiments a spray fixture encompasses a nozzle, but not visa-verse.

“Newtonian fluid” a fluid that obeys Newton's law of viscosity, represented as follows:

$$τ = μ \frac{dV}{dy}$$

or in other words, where the shear stress, τ (N/m²), is linearly proportional to the velocity gradient dV/dy, and where μ is dynamic viscosity (N s/m²), dV is unit velocity (m/s), and dy is unit distance between layers (m).

“Non-Newtonian fluid” as used herein includes fluids that contain suspended particles or dissolved molecules. This term may include, but is not limited to, Bingham fluids, pseudoplastic fluids, dilatant fluids, thixotropic fluids, and viscoelastic fluids. The term shall include, but is not limited to, fluids whose characteristics are represented by the Ostwald-de Waele equation as follows:

$$τ = {K ( \frac{dV}{dy} )}^{n}$$

where K (often in kg/ms²⁻ⁿ) and n (dimensionless) are constants determined by experimental fitting data. Generally, for pseudoplastic fluids, n is less than 1 and for dilatant fluids n is greater than 1.

“Labile” as used herein includes ingredients, components, particles, and/or fluids that are susceptible to changing state or losing a beneficial characteristic after prolonged contact with another ingredient, component, particle, and/or fluid. For example, dried or powdered milk is a labile ingredient that loses a preservative characteristic when subjected to prolonged moisture or water exposure.

“Metabolic input” as used herein includes any of the inputs or ingredients that are digestible, intended for animal and/or human consumption, or are otherwise capable of being utilized in metabolism. For example, this term includes, but is not limited to, food grade oils, probiotics, pre-biotics, antibiotics, in-feed enzymes, additives (e.g., processing additives), flavorants, colorants, odorants, or other food additives or compositions, solids with special properties such as chitosan, or combinations thereof. This term may also include edible polysaccharides (e.g., chitin), edible monosaccharides (e.g., glucose), edible triglycerides (e.g., palmitic acid or palm oil), glycerol, powdered edible inputs (e.g., powdered milk and/or cheese), or combinations thereof.

“Food application” as used herein includes any application and/or technique utilized in a process related to or associated with at least one of the following: seed treatments, planting applications, pest control, plant health, harvesting of food-related raw materials, food and ingredient transportation and storage, food and ingredient processing including preservation and flavor enhancement, food and ingredient packaging, encapsulation techniques in suspension or emulsion recipes (e.g., yogurt and other dairy products), shipping and storage applications, consumption preparations, and combinations thereof.

“Viscosity” as used herein includes dynamic viscosity measured at room temperature (e.g., 20° C.) unless specifically specified otherwise. In embodiments using a Bingham plastic or a fluid that can be represented by the Bingham plastic model, the term shall include plastic viscosity.

“High viscosity” or “highly viscous fluids” as used herein includes fluids having a viscosity within the range of 0.8 to 5 kg/m·s (800 to 5000 cP), inclusive. Examples of fluids having high viscosity include glycerol dispersions, suspensions, or emulsions. Corresponding yield stress, t, will vary depending on the fluid, but generally ranges from 10-200 Pa.

“Medium viscosity” with respect to fluids, includes a fluid having a viscosity within the range of 0.86×10⁻³ to 0.08 kg/m·s (0.86 to 80 cP), inclusive. Examples of fluids having medium viscosity include water and catsup. In some embodiments, a medium viscosity fluid is from 1 cP, inclusive, to 800 cP, exclusive.

“Low viscosity” with respect to fluids, includes a fluid having a viscosity within the range of 0.97×10⁻⁵ to 2.28×10⁻⁵ kg/m·s (0.0097 to 0.0228 cP). Examples of fluids having low viscosity include air, nitrogen, and Xenon. In some embodiments, a low viscosity fluid is from 0.0097 cP, inclusive, to 1 cP, exclusive.

“Emulsifying agent” as used herein includes a substance that has hydrophobic and hydrophilic properties, allowing dissolution of the substance in fatty or oily solutions and in aqueous solutions. The term shall encompass food related emulsifying agents, including but not limited to, agar, albumin, alginates (e.g., sodium alginate, potassium alginate, etc.), casein, ceatyl alcohol, cholic acid, desoxycholic acid, diacetyl tartaric acid esters, egg yolk, glycerol, gums (e.g., carob gum, guar gum, larch gum, etc.), processed seaweed or seaweed extracts (e.g., carrageenan, furcellaran, etc.), polysaccharides (e.g., konjac, soybean hemicellulose, pectin, cellulose, or combinations thereof), lecithin, mono- and diglycerides (e.g., glyceryl monostearate), esters of mono- and diglycerides (e.g., acetic acid esters, lactic acid esters, citric acid esters, etc.), phosphates (e.g., sodium polyphosphate, potassium polyphosphate, sodium triphosphate, pentapotassium triphosphate, dicalcium diphosphate, etc.), propylene glycol, and combinations thereof.

“Encapsulation” as used herein includes a method/process for distributing (e.g., entrapping) a first ingredient (e.g., active ingredient, pre- or probiotic, etc.) within a second ingredient (e.g., high viscosity fluid, carrier, amphiphilic component, or combinations thereof). In embodiments, this delivery of the first ingredient within a second ingredient may delay delivery of a limiting reactant, partially isolate the first ingredient, encircle a portion of the feedstock material including the first ingredient within a coating or a shell, affect a reaction rate of the first ingredient, and combinations thereof. For instance, a result of encapsulation may include improving a controlled release characteristic, delaying delivery until the first ingredient reaches an action or reaction site, improving a preservation characteristic (e.g., by providing a barrier between the first ingredient and one or more reactants), generating particles with a size of a few nanometers or millimeters, and combinations thereof.

“Amphiphile” as used herein means a molecule with both hydrophilic and hydrophobic properties.

It is an object of the inventive concepts disclosed herein to obtain improved delivery methods and apparatuses to improve upon existing food techniques, applications, mechanisms, and/or apparatuses. Improvements may include, but are not limited to, improved selectivity criteria, improved drop and spray emission patterns, improved spray nozzles, improved adjustability, improved delivery, improved mixing of immiscible ingredients, improved precision in labile ingredient separation and mixing, improved livestock feed application which may improve body weight gain (BWG) and/or feed conversion ratio (FCR), improved application to seeds, improved seed uptake, reduced energy associated with delivery and/or distribution, improved plant drying which may reduce a time required before harvest may occur, improve feedstock-to-surface spreading, improved uniform distribution on food or food related surfaces, other improvements as will be recognized by those skilled in the art, and combinations thereof.

In many of food techniques and/or applications, ingredients do not mix well together. In many of these techniques and/or applications, reaction times of ingredients must be limited to prolong product degradation. In many of these techniques and/or applications, ingredients must not be damaged by delivery mechanisms. In many of these techniques and/or applications effective delivery may be proportional to, or affected by, particle size. In many of these techniques and/or applications a batch or a continuous process is required for effective combinations. In most of these techniques and/or applications, it is desirable to reduce production costs associated with conventional feedstock delivery. In many of these techniques and/or applications, delivery mechanisms should not result excessive clogging at inlets or outlets or require excessive pulsation and vibrations to minimize the clogging. Further, these delivery mechanisms should be easily adjustable to deliver variable, desirable drop sizes, forms, particles, mists, or spray emission patterns.

In the food industry, raw ingredients often begin as seeds. Regardless of the end product (e.g., whether those seeds are the consumable product, or they are processed to form the consumable product and/or by-product), using apparatuses and methods to treat the seeds may improve seed size, moisture and/or treatment uptake, quality, stress-conditioning, vigor and viability (e.g., despite adverse weather conditions), other desirable seed characteristics, and combinations thereof.

Broadly, the inventive concepts disclosed herein are directed to a spray fixture selection, applicator selection, applicator configuration, and/or dimension adjustments based upon feedstock lability. Upon determining a level of lability associated with an emitted feedstock, a categorical determination may be made. Using the categorical determination and one or more additional feedstock and/or propellant characteristics, a spray fixture, applicator, application configuration, and/or dimension adjustment is selected. Accordingly, calibration, dimensional, operational, or fixture parameters may be derived based on the selection and desired lability, thermodynamic, fluidic, or kinematic properties.

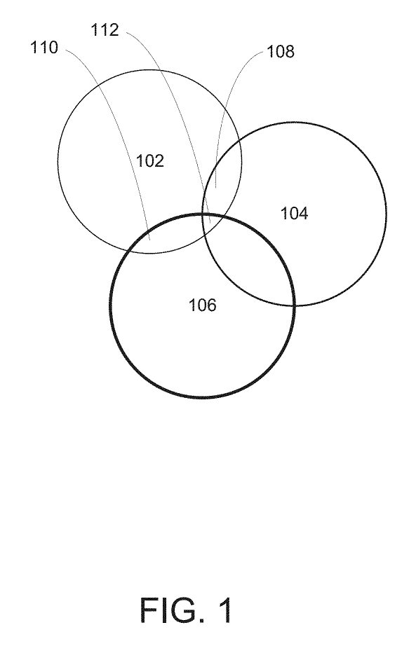

Referring now to FIG. 1, a Venn diagram depicts limiting selection or configuration criteria according to the inventive concepts of the present disclosure. For example, the first circle 102 represents all possible food applications, including but not limited to, seed treatment, planting, pest control, plant health, harvest and desiccation, processing, preservation, flavor enhancement, encapsulation, suspension, emulsion techniques, packaging, shipping, and storage applications. The second circle 104 represents a total number of possible distribution mechanisms, delivery mechanisms, spray fixtures, applicator configurations, and combinations thereof. The third circle 106 represents lability levels within lability reference frames. The overlapping region 108 represents distribution mechanisms, delivery mechanisms, spray fixtures, applicator configurations, or combination thereof that will operatively or correctly function for a specific food application. The overlapping region 110 represents a first number of applicable lability levels and reference frames for a specific food application. The overlapping region 112 represents a number of distribution mechanisms, delivery mechanisms, spray fixtures, applicator configurations, or combination thereof that will operatively or correctly function for the specific food application at the second number of applicable lability levels and reference frames (lower than the first number).

In embodiments, one or more characteristics of the feedstock or one or more characteristics of a feedstock material/ingredient are determined to begin the developing of limits for selection criteria. For example, the feedstock may include ingredients, components, or particles that are labile. Feedstock materials that are labile must be delivered and/or distributed with precision and care. For instance, an emulsion for a seed treatment application may be spray-dried for storage and later used in seed treatment prior to planting. During the spray drying process, a propellant (e.g., heated air or gas) may contain moisture, affecting the lability of the emulsion. If the propellant contains a high moisture content (e.g., 0.015-0.055 kg/m³), then contact with a highly labile ingredient should be minimized, or heating and/or evaporation should take place after the propellant comes in contact with the highly labile ingredient. These determinations may enable a selection of a proper applicator. For example, the applicator may maintain separation between the propellant and the highly labile ingredient until immediately preceding application (e.g., brought into contact within nozzle, or within spray fixture just before nozzle).

In embodiments, determining a feedstock characteristic to limit selection and/or configuration criteria may include determining costs or ease of production of one or more ingredients of the feedstock material. For example, a first ingredient (e.g., natural) may possess similar characteristics as a second ingredient (e.g., synthetic), but may cost more or may be more difficult to obtain. In such situations, often the second ingredient is used as opposed to the first to minimize overall costs, however, this may depend on other factors (e.g., environmental conditions, EPA regulations, etc.). In this regard, using the second ingredient may involve using fixtures that may operationally handle the lability characteristics of the second ingredient (e.g., most cost-effective to use a low-pressure system that can distribute a highly viscous fluid).

In embodiments, determining a feedstock characteristic may include determining mixing characteristics and/or desired flow rates for labile ingredients of the feedstock material. For example, feedstock materials may have a limit as to the amount of mixing that can occur during transport through a circular pipe, thus mixing characteristics are determined to limit the length of pipe used during transport. By way of another example, the feedstock material may need to be mixed further during flow from a hopper to a distribution mechanism. In either example, a desired flow rate of the feedstock material and mixing characteristics may be determined with respect to turbulent flow through a circular pipe according to a dispersion coefficient, such as in the relationship below:

D_{turbulent}=3.57\sqrt{fVD} (1)

where D_{turbulent} is a dispersion coefficient (e.g., axial dispersion coefficient), f is a friction factor, Vis velocity (e.g., average velocity of the fluid), and D is diameter (e.g., pipe diameter). In this regard, the flow rate may be determined using a second relationship (e.g., Equation (4), below).

In an exemplary embodiment, seeds treated, planted, coated, packaged, shipped, or otherwise prepared for consumption (e.g., by animal or human) may include one or more breeds of corn, including but not limited to, Cusco or Peru corn seeds, which may be used for snack foods (e.g., corn nuts) or for producing feed for livestock. It is noted that while embodiments may be discussed herein with respect to a specific food product (e.g., corn nuts), the inventive concepts will not be limited to this specific example, but will be recognized by those skilled in the art to be applicable to numerous food products and food applications.

### Seed Treatment and Planting

In embodiments, treatments may be applied to a corn seed prior to or during planting. For example, one or more fungicides, insecticides, or other treating material may be applied to the corn seed surface. For example, fludioxonil, neonicotinoids, or other compounds used as an insecticide in a feedstock material prior to or during planting. For instance, the other compounds may be any compound used to prevent, reduce, or eliminate insects affecting corn growth, such as aphids, billbugs, thrips, stink bugs, chinch bugs, flea beetles, nematodes, Pythium, or other such diseases, fungi, or pests.

In some embodiments, the seed treatment may aid in developing uniformity in size and shape of seeds for planting. For example, seed treatment applications may include seed pelleting.

In some embodiments, the seed treatment may include priming seeds with a high viscosity (e.g., low lability) ingredient. For example, corn seeds may be primed in a solution containing polyethylene glycol.

In some embodiments, a seed coating applicator may include a batch machine including a rotating drum and mixing baffles. The batch machine may include a first grate or a screen with a first diameter for a first type of seed, or a second grate or a screen for a second type of seed, which are sprayed and treated within the batch machine. In other embodiments, the seed coating applicator may include a continuous coating machine, with adjustable coating rates and adjustable product conveyor rates. In other embodiments, the seed coating applicator may include an atomized spray fixture within a closed system applicator.

In embodiments, the applicator is selected to apply a desired feedstock, which may include but is not limited to, one or more of: methyl N-(methoxyacetyl)-N-(2,6-xylyl)-DL-alaninate (e.g., metalaxyl), fludioxonil, a neonicotinoid, a phthalimide, a carboxin, a thiophosphoric acid ester (e.g., diazinon), ambectin, triticonazole, and combinations thereof.

In embodiments, one or more characteristics of the feedstock or one or more characteristics of a feedstock material/ingredient are determined to begin the developing of limits for applicator selection criteria and/or configuration criteria. For example, if the feedstock ingredient includes an insecticide as well as a biological or microbial growth regulators or plant-hormone based products, the mixing of ingredients must be done with precision and care with respect to the lability characteristics of the ingredients.

In embodiments, a feedstock characteristic is determined including a determination that a feedstock material is highly viscous (i.e., has low lability). For example, a seed suspension fluid used in distributing (e.g., directly planting into soil or spraying for planting onto prepared soil) seeds, may include treatment materials for treating the seeds. In some embodiments, the treatment materials may include microorganisms for treating the seeds prior to planting (e.g., inoculation during priming). In other embodiments, the seeds may be treated with a probiotic strain, including but not limited to, Bifidobacterium, Enterococcus, Lactobacillus, Bacillus, Pediococcus, Streptococcus, and/or combinations thereof. In some embodiments, an emulsion may be utilized to encapsulate the probiotic strain prior to delivery in order to enhance viability of the microorganism cells during metabolism and intestinal absorption. It is noted that the encapsulation process is not limited to seed treatment applications, but may also be used in other food applications (e.g., pest control and plant health; processing, preservation and flavor enhancement; etc.).

In embodiments, the determination that a feedstock includes a high viscosity fluid may include a determination that the feedstock should include an amphiphile. For example, the feedstock may be applied to a surface of a seed, and may include a surfactant or a wetting agent. In some embodiments, the surfactant includes, but is not limited to, sodium dodecyl benszenesulfonate, abietic acid, dimethyl ether of tetradecyl phosphonic, polyethoxylated octyl phenol, glycerol diester (diglyceride), sorbitan monoester, dodecyl betaine, N-dodecyl priridinium chloride. In some embodiments, the wetting agent includes a sulfo-carboxylix compound, including but not limited to, di-bis-ethyl-hexyl sulfosuccinate and di-bis (ethyl-hexyl) sodium sulfosuccinate. By way of another example, the feedstock may be applied to a seed in the form of an emulsion, thus, the amphiphile may include an emulsifier or an emulsifying agent. For instance, mono- and diesters of fatty alcohols (R—OH), neutralized by an alkaline hydroxide or a short amine may be used. In some embodiments, the emulsifier includes a diglyceride phosphoric acid (e.g., lecithin).

In embodiments, the amphiphile is anionic. In other embodiments, the amphiphile is cationic. For example, the amphiphile may include benzalkonium chloride (or a salt thereof). In other embodiments, the amphiphile is zwitterionic. For example, the amphiphile may include one or more fatty acid amides, amino acides, or betaines, including but not limited to, cocamidopropyl betaine, alkyl betaines, sulfobetaines, alkyl sulfobetaines, dilyceride amino phosphoric acid, and combinations thereof.

In embodiments, the determination that a feedstock includes a high viscosity fluid includes a determination that a feedstock includes a glycoside. For example, the feedstock may include a saponin.

In embodiments, the determination that a feedstock includes a high viscosity fluid includes a determination that a feedstock includes a water soluble polymer. For example, the feedstock may include poly(ethylene oxide), carboxylmethylcellulose, hydroxypropyl methylcellulose, and combinations thereof.

In embodiments, the determination that a feedstock includes a hexitol (e.g., ester of hexitol) or a cyclic anydrohexitol. For example, the feedstaock may include sorbitan, sorbitane (e.g., sorbitane mono stearate), and combinations thereof.

In an exemplary embodiment a feedstock characteristic is determined such that an ingredient of the feedstock does not mix well with another feedstock material/ingredient. For example, an emulsion may be termed a dispersion of two or more immiscible liquids in the presence of a stabilizing compound (e.g., emulsifier) and a seed planting application may involve the delivery and/or distributing of one or more emulsions.

In an exemplary embodiment, determining a feedstock characteristic may include determining an ability to obtain a desired coverage. This determination may be related to a determined viscosity of the fluid. For example, a seed or multiple seeds may be sprayed or mixed together with a seed suspension fluid (e.g., which could include a seed treatment fluid) as the planter/applicator moves across a field. In this regard, the desired coverage may be clumps or globules of seed suspension fluid and seed distributed per meter, per acre, per foot. For instance the viscosity of the fluid may be increased or decreased in order to adjust a number of seeds that are contained within the clump or globules that are distributed (e.g., sprayed) for planting a field, and the desired coverage may be two to three globules per meter.

In embodiments, limiting application selection criteria and/or configuration criteria may include determining that the applicator should include a spray fixture that incorporates a propellant (e.g., air-assist nozzle). The limiting of application selection criteria and/or configuration criteria may be based on propellant characteristics or propellant and feedstock combined characteristics. In this regard, the propellant characteristics and/or the propellant and feedstock combined characteristics may be determined with respect to a lability reference frame. For example, during operation, the feedstock may be determined to have a first velocity that is smaller than a distribution velocity (e.g., a desired rate at which the feedstock combined with a propellant leaves a nozzle). Using a viscosity lability reference frame, the propellant may need to have a specific viscosity (e.g., thereby adjusting velocity), or be within a range of viscosities, to make up the difference and propel the feedstock to obtain the desired distribution velocity. In this regard, the lability level of the propellant may be adjusted (e.g., increasing/decreasing pre-heat temperature, adding or removing a viscosity modifier, diluting or thickening the propellant and/or the feedstock, etc.) such that the propellant may propel a treated seed at a low energy and a low pressure (e.g., 1-15 psi). Thus the lability characteristics of the propellant may be adjusted to produce a specific velocity and/or viscosity (e.g., from 0.01 to 1 cP) in order to make up any detected differences between feedstock flow rates or distribution velocities and desired flow rates or desired distribution velocities. It is noted that velocity and/or viscosity may be related and/or determined using Reynolds number(s), friction factor(s), Bernoulli's equation, other relationships known in the art, and combinations thereof.

In some embodiments, the propellant may be selected based on lability characteristics of the feedstock. For example, if the feedstock is an emulsion and/or contains a probiotic strain such that little or no additional moisture may be added by a propellant in the delivery of the feedstock, then the propellant may be selected as nitrogen, or another inert gas, that is not air in order to reduce a moisture content. In other embodiments, air may be selected together with an additional process step (e.g., pre-heat) based on the lability characteristics of the feedstock, such that the moisture content of the air is minimized (e.g., via evaporation or other water removal) prior to combining with and propelling the feedstock.

In embodiments, limiting application selection criteria and/or configuration criteria may include configuring a spray applicator based on lability characteristics of the feedstock, the propellant, or the feedstock and propellant combination. For example, the type of applicator may be one of a planter, sprayer, extruder, or combination thereof determined based on the lability characteristics. By way of another example, a delivery tube type (e.g., pipe, slit, corrugated tubing, etc.), length, geometry, etc., may be determined using an equation/relationship including but not limited to, Bernoulli's equation, Reynolds No., friction factors, etc., which may take into account the lability characteristics.

It is noted that the lability characteristics may be a function of the application environment. Therefore, in some embodiments, determining lability characteristics may also include determining environmental factors, including but not limited to, humidity, air pressure, ambient temperature, air flow (e.g., wind speed), ventilation, and combinations thereof.

In some embodiments a determination may also be made as to whether a preexisting applicator (e.g., spray vehicle) may be retrofitted based on the lability characteristics. For example, a speed of the applicator vehicle (e.g., tractor/planter) and/or related components (e.g., gears connected to press wheel) may be determined in order to determine whether the applicator, components thereof (e.g., auger), the delivery mechanism, and the delivery tube for the desired application (e.g., planting) may safely distribute (e.g., without breaking or cracking) the seed for the intended application. In this regard, an applicator may include a power-take-off (PTO) drive and attachment. The emission form (e.g., drop patterns, fibers, mists, etc.) may also be determined in order to determine whether a pre-existing applicator may be retrofitted.

### Pest Control and Plant Health

In embodiments, lability characteristics of the feedstock, propellant, or feedstock and propellant combination are determined with respect to an ingredient used for pest control. For example, feedstock ingredients may include algicides, antimicrobials, biocides, miticides, fungicides, insecticides, nematicides, rodenticides, or combinations thereof. In this regard, a lability characteristic may include a combined lability characteristic such as viscosity and desired plant surface area coverage. For example, a desired coverage of a plant surface may be 50-100% plant or leaf surface area coverage for a specific amount of time (e.g., at least 12 to 24 hours). Using a high lability (e.g., low viscosity) solution together with the pesticide, herbicide, or foliar spray (i.e., as with conventional systems), may only result an obtainable coverage of 20% of plant or leaf surface area for a relatively short period of time (e.g., 5-10 minutes). Therefore, in some embodiments, a low lability solution (e.g., high viscosity feedstock) is used in a pest control application to provide an increased plant/leaf surface area coverage.

In some embodiments, the food application may include a plant health application that involves applying the feedstock material to the ground, or to an exterior or foliar portion of a plant. For instance, the plant may be corn, and the pesticide application may be a pre-emergent application of atrazine or glyphosate. In other embodiments, the plant health application is a post-emergent application.

### Harvest and Desiccation

In embodiments, an applicator is used to apply a desiccant to greenery at harvest time. This may reduce greenery processed (e.g., though a combine) and result a more efficient harvest. In embodiments, methods are disclosed for selecting a proper applicator for harvest and desiccation. For example, spray applicator criteria and/or configuration criteria may be limited by lability characteristics of the feedstock, propellant, or the feedstock and propellant combination.

In embodiments, lability characteristics of the feedstock, propellant, or feedstock and propellant combination are determined with respect to an ingredient used for harvest and desiccation. For example, feedstock ingredients may include pesticides and/or herbicides applied at, or prior to, harvest to improve plant dehydration, uniformity of ripening, weed and pest control for future crops, re-planting times, and combinations thereof. Such applications may also reduce time required before harvest, an amount of greenery present during harvest, strain on harvesters, and combinations thereof. In this regard, a lability characteristic may include a combined lability characteristic such as 1) a resistance to shear force within a viscosity lability reference frame, and 2) a resistance to dispersion within a drift lability reference frame, such that using the combined lability characteristic a desired plant/area of application is obtained without affecting surrounding areas. For example, a pesticide may have negative impacts on surrounding ecosystems or environments if it is carried beyond the plants for which they are intended, therefore, the combined lability characteristic may be used to limit spray applications to the desired plant/area of application. By way of another example, the lability characteristic may include a combined lability characteristic such as 1) a resistance to shear force within a viscosity lability reference frame, and 2) a resistance to dispersion within a water content lability reference frame, such that using the combined lability characteristic a desired plant/area of application is obtained without affecting nearby ground supply. For instance, if an emulsion is used to deliver the herbicide with low drift, then the feedstock/propellant may need to be selected with minimum moisture content (e.g., air or a gas instead of a liquid such as water) so as to reduce seeping or uptake into ground water reservoirs.

In some embodiments, a feedstock material with a low lability characteristic (e.g., high viscosity such as an emulsion) may be used together with the pesticide in order to reduce drift during application. For instance, the feedstock may include, but is not limited to, one or more of the following: sodium chlorate, glyphosate, carfentrazone-ethyl, cyanamide, cinidonethyl, pyraflufen-ethyl, and combinations thereof. In this regard, a mixture of glycol, or other high viscosity fluid, may be utilized to suspend, disperse, or emulsify the feedstock material for distribution in a low drift and/or a low energy application.

It is noted that a foliar spray for desiccation may include sprays that are generally detrimental to plant health. For example, a feedstock material may include, but is not limited to, glyphosate, diquat (e.g., 6,7-Dihydrodipyrido[1,2-a:2′,1′-c]pyrazinediium dibromide), paraquat (e.g., Dextrone-X), glufosinate, and combinations thereof.

In embodiments, limiting application selection criteria and/or configuration criteria for harvest and desiccation may include determining the characteristics of the feedstock material with respect to a lability reference frame. For example, the feedstock material may include a Diquat E-Pro herbicide. In this regard, the lability reference frame may be a combinational viscosity and temperature lability reference frame. For instance, the viscosity of the herbicide may be 1.6264 cP at ambient temperature, and a density of the herbicide may be 1.2006 g/mL. It is noted that other similar parameters may be obtained based on mixing and applicable testing with respect to the lability reference frame.

In some embodiments, the desiccation application applies the feedstock material to an exterior or foliar portion of a plant. For example, the plant may be corn, wheat, potatoes, etc., and the herbicide application may be a pre-harvest application of one of glyphosate, diquat (e.g., 6,7-Dihydrodipyrido[1,2-a:2′,1-c]pyrazinediium dibromide), paraquat (e.g., Dextrone-X), glufosinate, or combinations thereof.

### Processing, Preservation, and Flavor Enhancement

In embodiments, an applicator is used to apply a feedstock material/ingredient for processing, preservation, and flavor enhancement. For example, in some embodiments, proteins and/or probiotics may be used to coat ingredients (e.g., nut, corn seed, popcorn, Peruvian corn seed, etc.) intended for consumption and metaboliazation. The proteins and/or probiotics may be used to increase a nutritional value of the coated ingredients (e.g., to animals or humans). During the coating process, a propellant (e.g., air or gas) may contain moisture, affecting the lability of the proteins and/or probiotics. If the propellant contains a high moisture content (e.g., 0.015-0.055 kg/m³), then contact with the labile ingredient should be minimized in order to reduce exposure to stimulating environmental conditions and/or reactivation of microorganisms. In this regard, in some embodiments a temperature of the propellant is also adjusted to minimize affecting labile ingredients.

In embodiments, lability characteristics of the feedstock, propellant, or feedstock and propellant combination are determined with respect to an ingredient used for processing, preservation, and flavor enhancement. For example, feedstock ingredients may include sodium aluminosilicate, benzoyl peroxide, ethylenediaminetetraacetic acid (EDTA), bentonite, proteins or amino acids (e.g., lysine), potassium bromate, lecithin, yeast, actinobacteria, staphylococcus, halomanadaceae, enterobacter, mold, L. acidophilus, B. lactis, L. paracasei, Bifidobacterium PL1, L. bulgaricus, L. paracasei, B. breve, B. longum, B. infantis, L. rhamnosus GG, baking powder, baking soda, citric acid, lactic acid, ascorbic acid, butylated hydroxyanisole (BHA), butylated hydroxytoluene (BHT), citric acid, sulfites, tertiary butylhydroquinone (TBHQ), tocopherols, acetic acid, benzoic acid, natamycin, nisin, nitrates, nitrites, propionic acid, sorbic acid, sulfites, sulfur dioxide, anthocyanins, betacyanins, carotenoids, phenolics, synthetic food colorants, sweeteners (e.g., sucrose), flavorants, and/or combinations thereof. For example, if the feedstock includes lysine, then a lability characteristic may include a lability characteristic such as a resistance to shear force within a viscosity lability reference frame, such that using the lability characteristic the feedstock is delivered with a proper viscosity so the lysine remains on the surface of a corn nut or similar seed intended for consumption.

In some embodiments, a feedstock material with a low lability characteristic (e.g., high viscosity such as an emulsion) may be used together with an amino acid, probiotic, or other beneficial additive, in order to increase health benefits associated with consumption. For instance, the feedstock material may include, but is not limited to, one or more of the following: lycine, histidine, valine, isoleucine, leucine, methionine, phenylalanine, threonine, and tryptophan. In this regard, a mixture of glycerol, gelatin, pectin, or other high viscosity fluid, may be utilized to suspend, disperse, or emulsify the feedstock material for distribution in a low energy application.

In some embodiments, the determination that a feedstock includes a high viscosity fluid may include a determination that the feedstock should include an amphiphile. For example, the feedstock may be applied to a surface of a seed as a coating, and may include a surfactant or a wetting agent. In some embodiments, the surfactant includes, but is not limited to, dimethyl ether of tetradecyl phosphonic, glycerol diester (diglyceride), sorbitan monoester, dodecyl betaine, N-dodecyl priridinium chloride. In some embodiments, the wetting agent includes a sulfo-carboxylix compound, including but not limited to, di-bis-ethyl-hexyl sulfosuccinate and di-bis (ethyl-hexyl) sodium sulfosuccinate. By way of another example, the feedstock may be applied to a seed in the form of an emulsion, thus, the amphiphile may include an emulsifier or an emulsifying agent. For instance, mono- and diesters of fatty alcohols (R—OH), neutralized by an alkaline hydroxide or a short amine may be used. In some embodiments, the emulsifier includes a diglyceride phosphoric acid (e.g., lecithin).

In embodiments, limiting application selection criteria and/or configuration criteria for harvest and desiccation may include determining the characteristics of the feedstock material with respect to a lability reference frame. For example, the feedstock material may include a mustard slurry for flavor enhancement to an edible seed (e.g., corn nut; legume seeds, including but not limited to, beans, peas, or lentils; barley; pumpkin seed; chinquapin; sunflower, safflower, rapeseeds; oats; cocoa seeds; pine nuts; pecans; walnuts; brazil nuts; hazelnuts; macadamia nuts; almonds; pistachios; coffee beans; etc.). In this regard, the lability reference frame may be a combinational viscosity and temperature lability reference frame. For instance, within the viscosity lability reference frame, the plastic viscosity of the mustard slurry may be 0.25 kg/ms (250 cP) at ambient temperature (e.g., approximately 20° C.), and a yield stress of the mustard slurry may be 38 Pa. Thus, a spray fixture and application configuration may be selected to deliver the mustard slurry at a low energy or low pressure (e.g., 1-15 psi). It is noted that other similar parameters may be obtained based on mixing and applicable testing with respect to the given lability reference frame. By way of another example, the lability characteristic may be a resistance to degradation within a water content lability reference frame. For instance, if the feedstock for a corn nut coating includes a powered ingredient (e.g., milk or cheese), the powdered ingredient may need to consist of a specific moisture content during delivery and distribution in order to minimize spoiling prior to consumption. In this regard, the powdered feedstock may have a moisture (e.g., water) content of from 3-5%, thus a propellant may be chosen as a propellant having minimal moisture content. Further, an applicator configuration may be selected or limited to those applicators that do not combine ingredients until just prior to delivery to a surface of the seed. It is noted that dried milk is recommended for storage at a water activity (AW) level of 0.2, or a equilibrium relative humidity (ERH) of 20%; therefore, if an air supply is taken from a relatively humid environment, the air supply should be conditioned to remove moisture or another propellant should be selected.

In some embodiments, the food application may include a combinational food application such as 1) a flavorant application, and 2) a growth regulator application, wherein the feedstock material is applied to an exterior of a food ingredient and/or product. For instance, the food product may be corn nuts, and the feedstock may include a mustard slurry having probiotics, protein, other spices, food additives, and combinations thereof for the combinational food application. It is noted that in some embodiments, a food ingredient (e.g., corn seed) may be from a genetically modified plant, seed, or food source such that the ingredient (e.g., corn

KAM 16-1-2 seed) contains a higher nutritional value (e.g., higher protein content) than a natural or non-genetically altered ingredient.

In some embodiments, a seed food coating applicator (e.g., for processing, preservation, and flavor enhancement) may include a sprayer, extruder, coating and/or seasoning applicator (e.g., belt coater), belt-top applicators, or combination thereof, which is determined according to one or more lability reference frames and one or more lability characteristics (e.g., viscosity of as-applied coating and temperature of application). A delivery tube type (e.g., pipe, slit, corrugated tubing, etc.), length, geometry, etc., may be determined using an equation relating one or more lability characteristics to one another, including but not limited to, Bernoulli's equation, Reynolds No., friction factors, etc. The application environment may also be determined with respect to lability characteristics within a lability reference frame, including but not limited to, a humidity, pressure, temperature, air flow, ventilation, application temperature (e.g., spray or coating may be within an oven or an applicator with heated air flow), a time related to the application (e.g., how fast is the food product moving on a conveyor belt below), a desired and/or actual moisture content of a food product resulting from the spray application (e.g., moisture content of powdered milk), a surface area to be sprayed (e.g., belt and/or food surface area), an adsorption rate of the surface to be sprayed, a drying rate of the surface to be sprayed, and combinations thereof. A determination may also be made as to whether a pre-existing applicator (e.g., food sprayer) may be retrofitted to accommodate the lability characteristics. A speed of the applicator and/or related components (e.g., conveyor belt) may also be determined in order to configure the applicator for the desired application (e.g., processing, preservation, or flavor enhancement) according to the lability characteristics. In some embodiments, the emission form (e.g., drop patterns, fibers, mists, etc.) may also be determined with respect to a lability reference frame.

It is noted that some embodiments involve pre-conditioning (e.g., soaking in brine solution, blanching, etc.) the edible seed. It is further noted that some embodiments involve heating and/or roasting of the edible seed. In these embodiments, the feedstock material may be applied after the roasting to improve integration/infusion.

In some embodiments, the feedstock includes a preservative. For example, the preservative may include, but is not limited to, potassium sorbate, sorbic acid, propionic acid, calcium propionate, and combinations thereof. In some embodiments, the feedstock includes a nutritional enhancer. For example, the nutritional enhancer may include, but is not limited to, vitamin solutions, pre- or probiotics, other beneficial microorganisms, and combinations thereof. In some embodiments, the feedstock includes a flavorant. For example, the flavorant may include but is not limited to, mustard (e.g., slurry or powder), sucrose, dill, spices, and combinations thereof. In some embodiments, the feedstock may include a high viscosity fluid such as propylene glycol, honey, pectin, a demulcent, etc. In some embodiments, additional food additives may include, but are not limited to, herbs, spices, dairy ingredients, cocoa, coffee, tea, minerals, vitamins, amino acids, antioxidants, peanut kernel, sesame seed, soybean seed, dairy proteins, animal proteins, chilies, red chilies, peppers, black pepper, while pepper, red pepper, fruits, vegetables, maple flavor, maple syrup, agave, vanilla bean, vanilla flavor, mint, vinegar, sugar, honey, pomegranate, cider, orange juice or flavor, lemon, lime, cream, chocolate, garlic, caffeine, polyphenols, catechins, flavanols, tofu, caramel, jalapeno, ginger, edible acids, and combinations thereof.

In some embodiments, the edible seed is passed below one or more spray nozzles (e.g., on a conveyor belt), receiving the ingredient-feedstock mixture on a surface of the edible seed. In this regard, an adsorption rate of the edible seed surface may be determined to affect a distribution rate, size, form, or quantity of the ingredient-feedstock mixture. In other embodiments, the adsorption rate and a speed of the conveyor are both used in determining the distribution rate, size, form, or quantity. The adsorption rate may be determined using one or more diffusion equations. For example, models for unsteady state diffusion in a solid having one or more shapes may be determined according to the following:

$$\begin{matrix}{E}_{{ave}_{slab}} = {E}_{avgx} {E}_{{avg}_{y}} {E}_{avgz} = \frac{{c}_{{A}_{ave} - {c}_{{A}_{s}}}}{{c}_{{A}_{0} - {c}_{{A}_{s}}}} & ( 2 ) \\ E = {E}_{s} = \frac{{c}_{{A}_{s} - {c}_{A}}}{{c}_{{A}_{s} - {c}_{{A}_{0}}}} & ( 3 ) \\ E = {E}_{r} {E}_{x} = \frac{{c}_{{A}_{s} - {c}_{A}}}{{c}_{{A}_{s} - {c}_{{A}_{0}}}} & ( 4 )\end{matrix}$$

where Equation (2) applies to a slab shape, Equation (3) applies to a sphere, and Equation (4) applies to a cylinder, and E represents an E-value or an error function value, and c_{A}_{ₛ} is the steady state concentration of A, c_{A0} is the initial concentration of A, and C_{A}_{ₐᵥₑ} is an average moisture concentration after a period of time. It is noted that Equations (1), (2), and (3) are generally used with Fick's equation (Equation (5)) for diffusion, given according to the following:

$$\frac{\partial {c}_{A}}{\partial t} = {D}_{AB} ( \frac{{\partial }^{2} {c}_{A}}{\partial {x}^{2}} + \frac{{\partial }^{2} {c}_{A}}{\partial {y}^{2}} + \frac{{\partial }^{2} {c}_{A}}{\partial {z}^{2}} ) \qquad ( 5 )$$

In other embodiments, a drying rate of the surface may be calculated using equations similar to those given above. For example, if an emulsion is being sprayed directly onto a conveyor belt or similar surface (e.g., when forming edible films), the drying rate of a thin film spread across a surface may be a better equation/representation for determining desired parameters.

In some embodiments, various forms of drying relationships may be employed. For example, a drying relationship/equation may include the following:

$$\frac{\partial M}{\partial t} = D {∇}^{2} M \qquad ( 6 )$$

where t is time and M is the decimal moisture content (db) of an individual particle. It is noted that other forms of this equation/relationship may be as follows:

$$\begin{matrix}MR = {\sum }_{n = 1}^{z} {A}_{i} {e}^{- BiDt} & ( 7 ) \\ MR = \frac{6}{{π}^{2}} exp ( - Kt ) & ( 8 ) \\ MR = exp ( - kt ) & ( 9 )\end{matrix}$$

where D is the diffusion coefficient, t is drying time, Ai is the dimensionless characteristic of the drying object, Bi is the constants characteristic of the drying object (m⁻²), and K or k are drying constants (s⁻¹ or h⁻¹). In some embodiments, the K value for corn may be experimentally determined to be as follows:

$${K}_{corn} = 0.54 exp ( - \frac{5023}{{θ}_{abs}} ) \qquad ( 10 )$$

where K has units of s⁻¹ and θ is in ° R.

In some embodiments, the applicator includes an atomizing spray nozzle, capable of vaporization of ingredients (e.g., miscible and/or immiscible ingredients such as oily solutions, aqueous solutions, and combinations thereof) for improved mixing of ingredients prior to distribution to the edible seed.

It is noted that many factors may influence design considerations, including but not limited to, flow rates, delivery tube characteristics (e.g., diameter, friction factor, length, etc.), desired rate of application, characteristics of a moving means (e.g., rate of conveyor belt, length and/or width of conveyor belt, etc.), characteristics of feedstock, characteristics of propellant, combinations thereof, and others as will be recognized by those skilled in the art. For example, the edible seed may be one of many types of seeds, each with differing volumes.

In some embodiments, the edible seed is a corn seed. It is noted that the size, shape, and volume of corn seeds vary. In some embodiments, a volume of a corn seed is taken to fall within the range of 0.23 to 0.31 cm³, which would be much different for Peruvian corn seeds. It is noted that in embodiments used for applying a coating, a thickness of the coating or flavor slurry applied to the seed may vary. For example, the coating or thickness of flavor slurry may be from 0.16 to 0.64 cm ( 1/16^{th} to ¼ in.) thick. The desired thickness of the coating or flavor slurry will affect the delivery and distribution rate of an applicator (e.g., spray rate of a nozzle of a food sprayer). Assuming the corn seed and thicknesses given above, a desired flow rate of a distribution mechanism may be from 0.14 to 5.2 liters per second of feedstock-propellant mixture (e.g., with a density of a feedstock density combined with a propellant density). It is noted that the feedstock-propellant mixture may be an emulsion, suspension, or a dispersion, and may include a number of ingredients including solids or semi-solids, such that an exact density of the mixture will vary. However, it is noted that Example 1 (below) uses a density of glycerol, 1.2613 g/cm³, as an illustrative example, which use is not meant to be limiting.

In some embodiments, a surface roughness value of the pipe or tubing will vary depending on the type of pipe or tubing. In some embodiments, a friction factor plot for circular pipes may be used to determine a range of fanning friction factors (e.g., chemical engineer fanning friction factors). (See, for example, L. W. Moody, “Friction factors for pipe flow,” Trans. ASME 66:672 (1994)). In some embodiments, schedule 40 smooth pipe is used, for which a fanning friction factor range may be from 0.0056 to 0.0060. Properties of the propellant will vary depending on the propellant. For example, fluid densities and viscosities of air may include a density of air, 1.225 kg/m³ (0.076 lbm/ft³), and a viscosity of air of 1.81×10⁻⁵ kg/ms (0.0438 lb/fth) (at sea level and 15° C.). In some embodiments, a range of Reynolds numbers or a range of fanning friction factors, are used with known properties to determine a velocity or a range of velocities. In this regard, using the known pressure for the air supply (e.g., 1/16^{th} psi to 10 psi or from 1/20^{th} psi to 4 psi), and using a relationship between pressure, velocity, fanning friction factor (or Reynolds number), and pipe/tubing length, certain design factor unknowns are determined. For example, using a typical pressure-drop curve for a specific fluid in a pipe, a correlation between length of pipe/tubing and pressure gradient or pressure drop may be determined. By way of another example, known equations may be used. For instance, design unknowns may be found based on the following equation derived from Bernouli's Equation:

$$Δ P = 4 f \frac{Δ x}{D} ρ \frac{{V}^{2}}{2} \qquad ( 11 )$$

Equation (11) (or variations of it) may be first used to determine unkown properties of a propellant (e.g., air) and then used to determine unknown properties of the feedstock based on the known and determined properties of the propellant. In other embodiments, if a volume of coating or a desired thickness of coating to be placed on seeds having a known volume (or a volume falling within a range of known volumes), then the properties of the feedstock may be used (e.g., spray flow rate, pressure loss in a specific length and/or type of pipe/tubing, etc.) to determine the desired properties of the propellant (e.g., velocity, flow rate, etc.) and some unknown characteristics of the applicator (e.g., length of pipe/tube following point of contact).

In embodiments, limiting selection criteria and/or configuration criteria may include determining a length of the pipe/tubing used to deliver the propellant or the feedstock. For example, the length may be increased or decreased depending on a desired distribution pattern of a feedstock-propellant mixture (i.e., a factor in how far a droplet travels from a nozzle is a length of tubing or pipe) and one or more lability characteristics. In embodiments, an area (e.g., circular or annulus area) of an outlet of a nozzle may be increased or decreased depending on the desired distribution pattern of a feedstock-propellant mixture and the one or more lability characteristics.

It is also noted that expansions and contractions (e.g., fitting losses) must also be taken into account together with the one or more lability characteristics in limiting selection and/or configuration criteria (e.g., determining a required length of pipe/tubing).

### Encapsulation, Suspension, and Emulsion

In embodiments, an applicator is used to apply a feedstock material/ingredient for encapsulation, suspension, and emulsion applications. For example, in some embodiments, ingredients may be brought together with an encapsulating ingredient to improve shelf-life, may be combined with a solid or semi-solid to form a colloidal suspension, or may be immiscible and brought together with an emulsifying agent to create an emulsion (e.g., yogurt). In this regard, using apparatuses and methods disclosed herein to combine and deliver feedstock including metabolizable inputs, nutrients, vitamins, emulsifying agents, immiscible ingredients, flavor enhancers, and/or combinations thereof, may improve an ability to deliver and apply low lability (e.g., highly viscous) fluids, an ability to control rates of reaction (e.g., according to a reactivity lability reference frame), uniformity of ingredient or food surface coverage, flavor profile of flavored snack foods (e.g., according to a gustatory lability reference frame), moisture control (e.g., as with combining and/or applying humectants according to an HLB lability reference frame), a delivery and application of stabilizers or thickeners (e.g., pectin, gelatin, gums, etc.), mixing of immiscible ingredients (e.g., as with emulsifications), and combinations thereof. In some embodiments, such applications may also reduce energy requirements for delivery and application of viscous fluids and/or emulsions.

In embodiments, lability characteristics of the feedstock, propellant, or feedstock and propellant combination are determined with respect to an ingredient used for encapsulation, suspension, and emulsion applications. For example, feedstock ingredients may include materials, compounds, or particles that have a high level of lability. For instance, labile ingredients including proteins, pre- or probiotics, beneficial microorganisms, or combinations thereof, may be used in an encapsulation application to reduce stress or strain during delivery and/or distribution.

In some embodiments, labile ingredients may be encapsulated for consumption benefits and/or delayed metaboliazation. For example, the labile ingredients may be used to increase a nutritional value of coated ingredients (e.g., edible seeds, animal feed, etc.) or of a feedstock (e.g., dairy product) intended for animal or human consumption.

In some embodiments, the labile ingredients may be used in many pharmaceutical applications such as drug delivery and controlled release and in medical technology such as wound and burn dressings or surgical treatment (e.g., creams), dermatitis and fungal infections, contact lens, bacteriostat and fungistat and bone disease, biotechnology applications such as membranes, biocatalysts, enzyme immobilization, protein separation, cell immobilization, food products, preservatives, fat absorption animal feed additives, metal-chelating processes such as absorption of transition metal ions such as copper, chromium, lead, silver and so on, agricultural products such as timed-release, seed coating, foliar application, paper products, and combinations thereof. During an encapsulating process, a propellant (e.g., less viscous fluid) may contain reactants or components (e.g., water content) that negatively affect the lability of an ingredient of the feedstock (e.g., proteins, pre- or probiotics, etc.). For instance, if the propellant contains a high moisture content (e.g., 0.015-0.055 kg/m³), and is brought into contact with proteins and/or probiotics, then the contact may need to be minimized in order to reduce exposure to stimulating environmental conditions and/or reactivation of microorganisms. Thus, the selection criteria and/or configuration criteria of the applicator and/or the spray fixture may be adjusted based on the lability characteristics. In some embodiments, adjusting a configuration criteria of an applicator may include adjusting a temperature (e.g., pre-heat) of the propellant to minimize its effect on labile ingredients.

In embodiments, limiting application selection criteria and/or configuration criteria for encapsulation, suspension, and emulsion applications may include determining the characteristics of the feedstock material with respect to a lability reference frame. For example, the feedstock material may include any one of sodium aluminosilicate, benzoyl peroxide, ethylenediaminetetraacetic acid (EDTA), bentonite, proteins, potassium bromate, lecithin, yeast, actinobacteria, staphylococcus, halomanadaceae, enterobacter, mold, L. acidophilus, B. lactis, L. paracasei, Bifidobacterium PL1, L. bulgaricus, L. paracasei, B. breve, B. longum, B. infantis, L. rhamnosus GG, baking powder, baking soda, citric acid, lactic acid, ascorbic acid, butylated hydroxyanisole (BHA), butylated hydroxytoluene (BHT), citric acid, sulfites, tertiary butylhydroquinone (TBHQ), tocopherols, acetic acid, benzoic acid, natamycin, nisin, nitrates, nitrites, propionic acid, sorbic acid, sulfites, sulfur dioxide, anthocyanins, betacyanins, carotenoids, phenolics, synthetic food colorants, sweeteners (e.g., sucrose), flavorants, and/or combinations thereof. An emulsifying agent may be utilized to suspend, disperse, or emulsify the feedstock material for distribution in a low energy/pressure application.

In some embodiments, the determination that a feedstock includes a low lability within a viscosity lability reference frame (e.g., high viscosity fluid) may include a determination that the feedstock should include an amphiphile. For example, the feedstock may be applied to a surface of a seed, and may include a surfactant or a wetting agent. In some embodiments, the surfactant includes, but is not limited to, dimethyl ether of tetradecyl phosphonic, glycerol diester (diglyceride), sorbitan monoester, dodecyl betaine, N-dodecyl priridinium chloride. In some embodiments, the wetting agent includes a sulfo-carboxylix compound, including but not limited to, di-bis-ethyl-hexyl sulfosuccinate and di-bis (ethyl-hexyl) sodium sulfosuccinate. By way of another example, the feedstock may be applied to a seed in the form of an emulsion, thus, the amphiphile may include an emulsifier or an emulsifying agent. For instance, mono- and diesters of fatty alcohols (R—OH), neutralized by an alkaline hydroxide or a short amine may be used. In some embodiments, the emulsifier includes a diglyceride phosphoric acid (e.g., lecithin).

In some embodiments, the amphiphile is anionic. In other embodiments, the amphiphile is cationic. For example, the amphiphile may include benzalkonium chloride (or a salt thereof). In other embodiments, the amphiphile is zwitterionic. For example, the amphiphile may include one or more fatty acid amides, amino acids, or betaines, including but not limited to, cocamidopropyl betaine, alkyl betaines, sulfobetaines, alkyl sulfobetaines, dilyceride amino phosphoric acid, and combinations thereof.

In some embodiments, a lability characteristic is determined with respect to a lability reference frame that uses another compound or feedstock ingredient (e.g., water/oil). For example, an emulsion may be termed a dispersion of two or more immiscible liquids in the presence of a stabilizing compound (e.g., emulsifier) and a processing, preservation, or flavor enhancing application may involve the delivery and/or distributing of one or more emulsions. It is noted that a hydrophobic portion of an emulsifying agent contributes to dissolution of that portion of the agent into the oily phase of the feedstock material, while a hydrophilic portion of the emulsifying agent contributes to dissolution of that portion of the agent into an aqueous phase of the feedstock material. In this regard, a stable oil-in-water emulsion (e.g., low lability) may be generated, where lability may be related to an HLB value. In some embodiments, an emulsion is generated without an in-line static mixer.

It is noted that the Food and Drug Administration (FDA) has specific rules, regulations, and/or requirements, which may vary depending on intended use. For example, at least four different categories may exist with different guidelines for each category and the use of probiotics. For instance, an intended use may be a “food”, “medical food”, “dietary supplement”, “drug” or “biological product”, where a medical food is intended for enteral use for disease management through formulated administration, a dietary supplement supplements the diet and is not conventional food, a drug is for treating or mitigating a disease, a biological product may contain a virus or serum or toxin for treating a disease, and foods are articles intended for food. Biologicals and new drugs may be regulated under Federal Food, Drug, and Cosmetic Act. 21 U.S.C. § 201(p) (2007); dietary supplements may be regulated under Federal Food, Drug, and Cosmetic Act. 21 U.S.C. § 343(r)(1)(B) (2007); and food or ingredients for food may be regulated under Federal Food, Drug, and Cosmetic Act. 21 U.S.C. § 321(s) (2007) or § 342(a)(2)(C)(i) and § 349. In some embodiments, concentrations of ingredients and/or feedstock applied to edible seeds, food surfaces, applicator surfaces (e.g., conveyor belt), raw ingredients, and combinations thereof, may be measured in order to determine compliance with one or more rules, regulations, and/or guidelines.

### Packaging, Shipping, and Storage

In embodiments, an applicator is used to apply a feedstock material/ingredient for packaging, shipping, and storage applications. For example, in some embodiments, ingredients may be brought together for sealing a packaging, shipping, or storage container, or portions therof (e.g., lid, tabs, entire container, etc.). In this regard, using apparatuses and methods disclosed herein to combine and deliver feedstock including binders, adhesives, glues, mucilage, thermoplastics, pastes, and/or combinations thereof, may improve an ability to deliver and apply highly viscous fluids, an ability to control rates of reaction, uniformity of adhesive application to packaging containers, shelf-life, moisture control (e.g., as with sealed containers), mixing of immiscible ingredients (e.g., as with emulsifications), and combinations thereof. Such applications may also reduce energy requirements for delivery and application of viscous fluids and/or emulsions.

In embodiments, lability characteristics of the feedstock, propellant, or feedstock and propellant combination are determined with respect to an ingredient used for packaging, shipping, and storage applications. For example, feedstock ingredients may include materials, compounds, or particles that have a high level of lability. For instance, labile ingredients susceptible to hydrolysis and/or saccharification, may be delivered to minimize contact with water, moisture, fungi, and/or bacteria. The labile ingredients may be used to create an adhesive.

In embodiments, limiting application selection criteria and/or configuration criteria for packaging, shipping, and storage applications may include determining the characteristics of the feedstock material with respect to a lability reference frame. For example, the feedstock material may include an ingredient that has low lability (e.g., highly viscous). For instance, the feedstock material may include any one of poly(vinyl acetate) (PVA), plasticizers (e.g., glycerol), polylactic acid (e.g., polylactide), polyethylene (e.g., low- or medium-density polyethylene), polypropylene, ethylene-vinyl-acetate, a mucilage, and/or combinations thereof. A mixture of the feedstock, a surfactant, or other ingredients, may be utilized to suspend, disperse, or emulsify the feedstock material and constructively combine it with a propellant resulting distribution in a low energy/pressure application.

In some embodiments, the feedstock material may include a medium viscosity fluid. For example, ingredients such as the poly(vinyl acetate) (PVA), plasticizers, polylactic acid (e.g., polylactide), polyethylene (e.g., low- or medium-density polyethylene), polypropylene, ethylene-vinyl-acetate, a mucilage, and/or combinations thereof, may be or may include a medium viscosity fluid. For instance, soybean glues have a very large range of operating viscosities, ranging from 500 cP to 75,000 cP. Accordingly, if a soybean glue is used as a feedstock material having a viscosity of 500 cP, then it may be characterized as a medium viscosity fluid, as disclosed herein. In contrast, if the feedstock material includes a soybean glue that has a viscosity of 2,000 cP, then it may be characterized as a high viscosity fluid, as disclosed herein.

In embodiments, a feedstock characteristic is determined including a feedstock material consisting of particles having a specific size. For example, particles may be the size of corn seeds, cabbage seeds, or onion seeds. By way of another example, a particle size may be expressed in terms of surface area. For instance a particle size of soybean meal used for adhesives may include a surface area of between 3,000-6,000 cm2/g, or in other words 97% is able to pass through a 325-mesh screen. In other embodiments, mesh numbers of 40, 60, and 80 (e.g., British standard sieves) may be used.

In some embodiments, determining a feedstock characteristic may include determining an ability to obtain a desired coverage according to a lability reference frame. For example, it may be desired that the desired area be covered, or feedstock applied, without dripping or running. This determination may be related to a viscosity lability reference frame. For instance, a food container may be sprayed with a feedstock containing an adhesive as the food container moves below the sprayer on a conveyor belt. In this regard, the desired coverage may be 0.1-50.0% conatainer surface area coverage. Through the use of a high viscosity fluid as the adhesive, or as a portion thereof, the desired coverage may be obtained without dripping and/or running. In contrast, a low viscosity fluid used in an applicator may not provide as much surface area coverage without running/dripping because the low viscosity fluid adheres less to the packaging container surface as compared to an application using a high viscosity fluid. Therefore, it is desirable to use high viscosity fluids for desired coverage without dripping/running. In some embodiments, a surfactant may be used to improve the spreading and/or distribution of the spray over a surface of a package or a food container.

In some embodiments, the propellant may be selected based on a degradation lability characteristic of the feedstock with respect to a water content lability reference frame. For example, if the feedstock includes a powered ingredient (e.g., soy protein with protein density index (PDI) of about 90), the powdered ingredient may need to consist of a specific moisture content during delivery and distribution in order to obtain a desired viscosity, minimize spoiling, and/or minimize activation prior to use. In this regard, the powdered feedstock ingredient may be relatively moisture-free, thus the propellant may be chosen as a propellant having minimal moisture content. In this regard, soy powder or soy flour is recommended for storage at moisture contents of below 9%; therefore, if an air supply is utilized as a propellant and is taken from a relatively humid environment, the air supply should be conditioned to remove moisture, or another propellant should be selected.

In embodiments, determining a feedstock characteristic may include determining a combined characteristic of feedstock ingredients. For example, a combined characteristic may include a type, size, and composition of a micelle. By way of another example, the Feedstock may include one or more reactants, r_{A}, r_{B}, . . . r_{N}, and in some embodiments it may be desirable to combine the one or more reactants (e.g., in the delivery mechanism, or a portion thereof) with each other prior to being propelled out a distribution mechanism by the propellant so as to induce a chemical reaction and a emission (e.g., spray) having a specific concentration of a reactant and/or product. In some embodiments, it may be necessary to determine a mole balance of the chemical reaction, which for a steady state, tubular reactor (e.g., delivery tube) may be determined as follows:

$$\frac{{dF}_{j}}{dV} = {r}_{j} \qquad ( 12 )$$

It may also be necessary to determine at what point reactants are combined in order to produce a decomposition, combination, or isomerization of a reactant. To determine this point, a chemical reaction volume, V₁, necessary for the decomposition, combination, or isomerization of a particular reactant, rA, may be determined in order to ascertain the point at which reactants are combined. For example, the chemical reaction volume, V₁, may be determined as follows:

$${V}_{1} = {\int }_{FA 0}^{{F}_{A 1}} \frac{{dF}_{A}}{{r}_{A}} = {\int }_{{F}_{A 1}}^{{F}_{A 0}} \frac{{dF}_{A}}{- {r}_{A}} \qquad ( 13 )$$

where V₁ may be characterized as a volume necessary to carry out a reaction such that an incoming flow rate, F_{A0}, is reduced to a specific value, F_{A1}, which by the nature of a chemical reaction, is also the volume necessary for a molar flow rate for generating a product (e.g., isomer, etc.).

### Lability Characteristics

In embodiments, lability characteristics are determined with respect to one or more lability reference frames. For example, viscosity of a feedstock ingredient may be a first lability reference frame, such that a first lability characteristic determined with respect to the viscosity lability reference frame includes a shear stress. In some embodiments, a viscometer (e.g., Brookfield viscometer), a rheometer (e.g., for determining rheological viscosity), or a viscotherm may be utilized in determining an amount or degree to which a fluid may be altered after receiving force (e.g., lability level in response to shear stress). It is noted that a drift lability reference frame may be closely related to the viscosity lability reference frame, except that the shear stress may be a result of a variable wind speed and also gravitational forces may be accounted for in the drift lability reference frame. By way of another example, a second lability reference frame may include a volatility reference frame, such that a second lability characteristic determined with respect to the volatility reference frame includes at least a boiling point and/or temperature. By way of yet another example, a third lability reference frame may include a temperature lability reference frame. To determine a lability characteristic in the third lability reference frame one or more devices (e.g., thermometer, thermocouple, UV spectrometer, etc.) may be used together with one or more established procedures (e.g., X-ray chromatography, differential scanning calorimetry curves, Native-PAGE and SDS-PAGE analysis, and combinations thereof) to determine a lability level within the third lability reference frame. By way of yet another example, a fourth lability reference frame may include a pH lability reference frame. To determine a lability characteristic in the fourth lability reference frame one or more devices (e.g., pH meter, litmus paper, pH indicator, etc.) may be used together with one or more established procedures (e.g., measuring electrical potential dfiferences between pH electrode and reference electrode) to determine a lability level within the fourth lability reference frame. By way of yet another example, a fifth lability reference frame may include a combinational lability reference frame, combining lability characteristics from two or more lability reference frames. It is noted that the lability reference frames are not limited to the examples above, but are intended to encompass all lability reference frames that will be recognized by those skilled in the art.

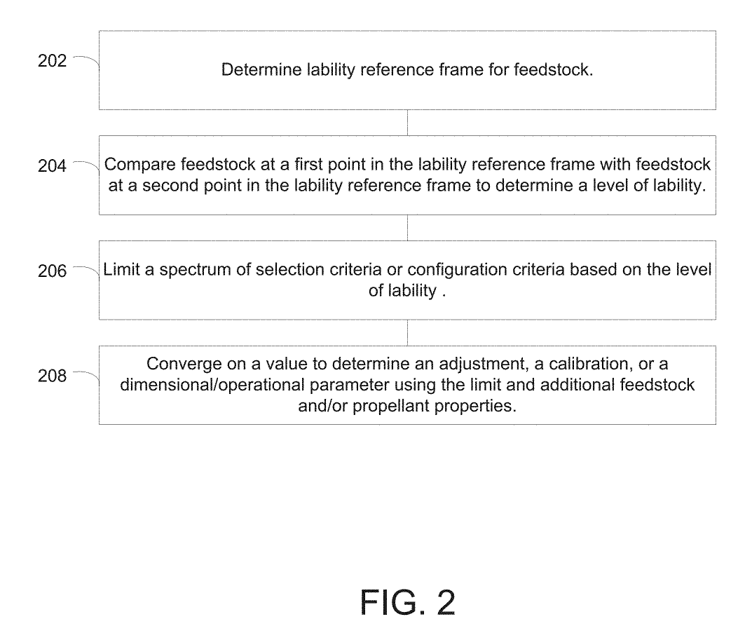

Referring now to FIG. 2 an exemplary embodiment of a method 200 according to the inventive concepts disclosed herein may include one or more of the following steps. For example, the method 200 may be a method for labile-based spray and emission for food applications.

Step 202 may include determining a lability reference frame for feedstock. For example, the lability reference frame may include, but is not limited to, lability with respect to pressure, temperature, pH, other reactants (e.g., chemical reactivity), an emulsion (e.g., how long feedstock stays as an emulsion), a resistance to shear or tensile stress, kinematic effects, exposure time, emission form (e.g., drops, mists, fibers, nan-particles, etc.), a reference frame with respect to another element or compound (e.g., water—hydrophobic, hydrophilic, or amphiphilic; or fat—such as lipophilic), and intermolecular forces (e.g., has a bonded by a covalent bond as opposed to bonding by Van der Waal's forces). For instance, a first lability reference frame may be temperature used in the context of applying biologicals to seeds for seed treatment. For instance, if a first lability level of lysine is determined at a temperature of 60 degrees Celsius (60° C.), a second lability level of lysine may be determined at 80° C. or 100° C., such that the second lability level is greater than the first lability level. Multiple levels between the first lability level and the second lability level, inclusive, may be considered a lability reference frame.

In some embodiments, the lability reference frame is a combinational reference frame. For example, the lability reference frame may be based on a combination of two or more of pressure, temperature, pH, other reactants (e.g., chemical reactivity), a resistance to shear or tensile stress, kinematic effects, intermolecular forces. For instance, the lability level of lysine at 60° C. and a pH of 10 may be less than the lability level of lysine at 100° C. and a pH of 10.6. By way of another example, a seed (e.g., corn nut) may be coated with an amino acid (e.g., lysine), which is intended as a metabolic input (e.g., intended to be eaten). In this regard, the combinational lability reference frame may include a combination of temperature, pH, and exposure time associated with an animal/human digestive tract.

In some embodiments, the lability reference frame is determined for an as-applied feedstock. For example, the lability reference frame may be determined for feedstock ingredients as they are intended to be applied to a seed for a seed coating or a seed treatment. By way of another example, the lability reference frame may be determined for feedstock ingredients with respect to the emission form at which they are intended to be applied. For instance, the lability reference frame may include determining a lability level within the reference frame of fibers, drops, mists, extrusions, emulsions, and other emission forms. For instance, drops and mists may be more labile than fibers, extrusions, and emulsions due to characteristics (e.g., exposed surface area, amount of unexposed ingredients, etc.) of the form in which they are emitted. In other embodiments, the lability reference frame is determined for an as-distributed feedstock. For example, one or more ingredients may be added to a hopper or a container for distribution to a spray fixture. In this regard, the lability reference frame may be determined with respect to the ingredients to be distributed—either before mixing or after mixing.

In some embodiments, the lability reference frame is determined for a combined feedstock and propellant mixture. For example, lysine may be an ingredient of a feedstock to be applied in a seed treatment application, and air or nitrogen may be used as a propellant to aid in the distribution, delivery, emission, or spray of the lysine to a seed for the seed treatment.

A step 204 may include comparing feedstock at a first point in the lability reference frame with feedstock at a second point in the lability reference frame to determine a level of lability. For example, an ingredient of the feedstock may be applied at a pH of 6-8 (e.g., average pH of rumen is approximately 6-7, while saliva has a pH value of approximately 8), such that the lability reference frame may be pH. In this regard, the lability reference frame may have multiple points determined with respect to an ingredient of the feedstock. For instance, the lability reference frame may include pH points of approximately 1.5 to 11.0, such that comparing the feedstock at a first point in the lability reference frame with feedstock at a second point in the lability reference frame may include comparing a feedstock ingredient such as lysine at the as-applied pH level of approximately 6-8 to lysine in a solution/medium having a pH of approximately 2-3 (e.g., average pH of abomasum is approximately 2-3) in order to determine the level of lability of the feedstock ingredient. In this regard, the lysine at a pH of 6-8 may be categorized to a low lability level. In contrast, lysine at a pH of 2-3 may be compared to lysine at a pH of 6-8, and may be categorized to a high lability level. Categorical levels of lability may include, but are not limited, high lability, medium lability, and low lability.

In some embodiments, the level of lability may be determined by comparing a feedstock ingredient at a first combinational point in the lability reference frame with a second combinational point in the lability reference frame. For example, a first amino acid (e.g., lysine) may be applied to a metabolic input already high in a second amino acid (e.g., seed/nut may already be high in arginine), and a lability level may be a high lability level at a temperature of 20° C. and a moisture content of greater than approximately 0.01 kg/m³, a medium lability level at a temperature of 20° C. and a moisture content of approximately 0.003 kg/m³ (e.g., about 30% relative humidity) to 0.01 kg/m³, inclusive, and a low lability level at a temperature of 20° C. and a moisture content of less than approximately 0.003 kg/m³.

In some embodiments, geographical region with effects including moisture content and temperature may be accounted for when determining lability level. For example, a corn nut receiving a seed treatment in a geographical region that has a relatively low moisture content (e.g., desert), may be associated with a feedstock having a different lability level than the same feedstock used for a similar corn nut receiving the same seed treatment in a geographical region with relatively high moisture content (e.g., coastal region).

In some embodiments, determining the level of lability in Step 204 may include determining that another feedstock ingredient is needed for the food application. For example, if the lability reference frame is a water-based lability reference frame, and the level of lability of a feedstock ingredient is determined to be a high level of lability (e.g., hydrophilic), then the Step 204 may include a determination that an emulsifying agent should be added as another feedstock ingredient.

A step 206 may include limiting a spectrum of selection criteria of distribution mechanisms, delivery mechanisms, spray fixtures, applicator configurations, and combinations thereof for food applications based on the level of lability determined in step 204. For example, for a food application such as seed treatment, a large number of applicators and/or spray fixtures may be available to perform the seed treatment, where each applicator or spray fixture may be associated with its own selection criteria. In this regard, the level of lability within the specific lability reference frame may limit the selection criteria or configuration criteria sufficiently such that a particular applicator or a particular spray fixture may be chosen for the seed treatment. For instance, a feedstock ingredient having a low level of lability within a viscosity lability reference frame (i.e., meaning the ingredient is highly viscous) may limit the spectrum of selection criteria such that a spray fixture having a circular arc opening may be selected for the seed treatment.

In some embodiments, the Step 206 may include limiting a spectrum of configuration criteria based on the lability level. For example, an applicator such as a fluid drilling applicator may be selected based on the level of lability for the specific lability reference frame determined in Step 204. For instance, the low-level lability ingredient within the viscosity lability reference frame (e.g., highly viscous ingredient), may be best delivered using a low pressure configuration, such as with an applicator that has an auger configuration like those used in fluid drilling applicators. By way of another example, a lability reference frame may be a combinational lability reference frame, wherein a lability level is determined with respect to at least three factors. The three factors may be chemical reactivity/structure, absorption rate, and metabolic interaction/conversion. In this regard, the lability level may be associated with a toxicity level or a toxicity lability reference frame such that specific food applications may be eliminated based on the lability level of toxicity. For instance, if a substance is highly labile within a toxicity lability reference frame (i.e., where toxicity level and lability level are directly correlated), then food applications such as processing, preservation, packaging, encapsulation, or any application that may present a direct health hazard if the highly labile ingredients were metabolized, can be eliminated or limited. In other words, the selection criteria would be programmed logically to remove those food applications from possible selections for the high-labile level ingredients. This would leave seed treatment, harvest and desiccation, and possibly shipment (e.g., container sealant for containers that are used to ship individual product packages such as a corn nut pouch) as potential food applications.

A step 208 may include converging on an adjustment parameter, a calibration parameter, a dimensional parameter, an operational parameter, or combination thereof using the limits determined in Step 206 and using additional feedstock properties. For example, the limit in Step 206 may help select or configure a particular spray fixture for a specific food application. Then, using an additional feedstock property such as a velocity of a sprayed feedstock or a flow rate of a sprayed feedstock, an adjustment to a nozzle of the spray fixture may be converged upon. In this regard, a feedback controller (e.g., proportional-derivative (PD), proportional-integral (PI), or proportional-integral-derivative (PID)) may be used to monitor feedstock properties such as flow rates and velocities at a first set-point value (e.g., initialization value). Based on the monitored values, a second set-point value may be converged upon. Using the second set-point value an adjustment value, calibration parameter, a dimensional parameter, an operational parameter, or combination thereof may be determined. For instance, if the spray fixture has a nozzle with an opening that is similar in shape and design to an annulus opening, an initial volume may be determined by solving a first annulus relationship V1=πh(R²-r²). This initial volume, V1, in some embodiments, may be an initial set-point. Based on monitoring the volumetric flow rate, or based on calculated or desired set-points for a desired flow rate, the initial volume may be reduced by rotating an outer (or an inner) cylinder associated with the annulus opening, such that a second volume V2 may be converged upon based on the monitored and calculated values of flow rates using a second annulus relationship, V2=πh(R²-r²)*(θ/360° (e.g., for an annulus segment). It is noted that statistical analysis, regression models, and optimization models (e.g., Monte Carlo method) may be used to converge on the adjustment value, calibration parameter, a dimensional parameter, an operational parameter, or combination thereof.

In some embodiments, a seed treatment application includes applying seed treatment material to seeds within a seed hopper and delivering the treated seeds using an auger delivery system directly to the soil for planting. In this regard, converging on an adjustable parameter may include determining the auger speed and nozzle opening dimension(s). For example, delivering a corn seed to a furrow should not involve cracking, breaking, or applying excessive pressure during the delivery. Accordingly, in embodiments, a fluid drilling delivery mechanism is disclosed, which may include an auger having threads designed for separating one, two, or more seeds from a feedstock material via a shearing surface of the thread for delivery to a distribution mechanism. In embodiments, the delivery and distribution includes singulated seed delivery and distribution together with a portion of feedstock material surrounding the seed to be delivered and distributed.

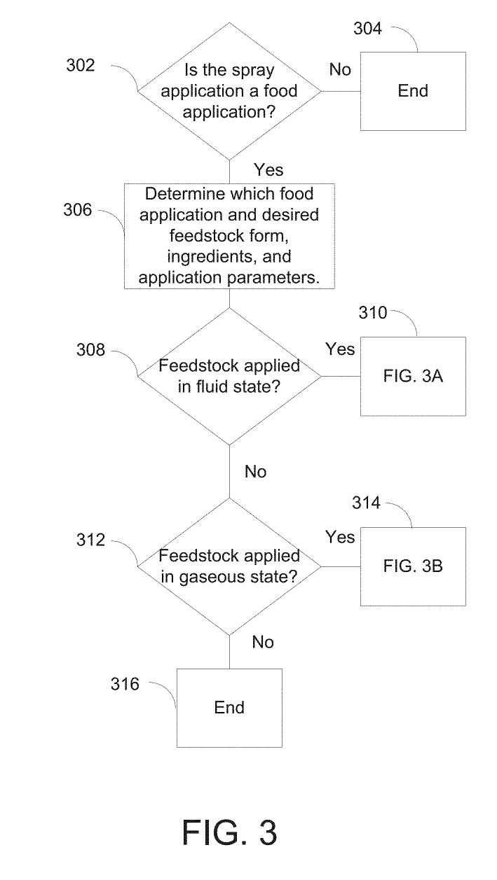

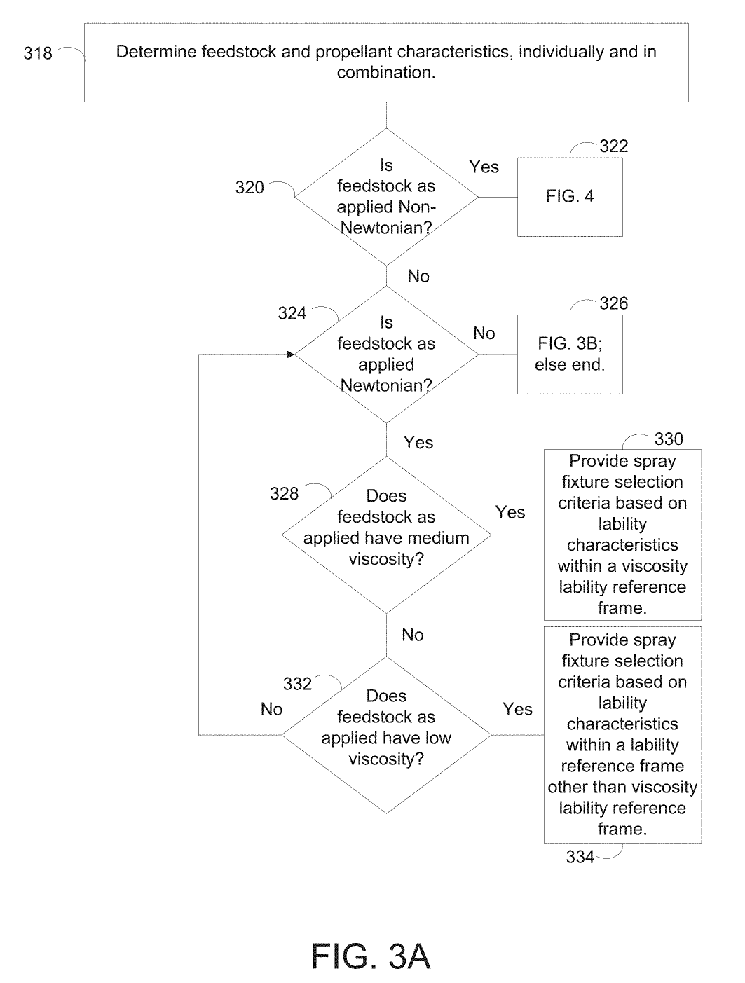

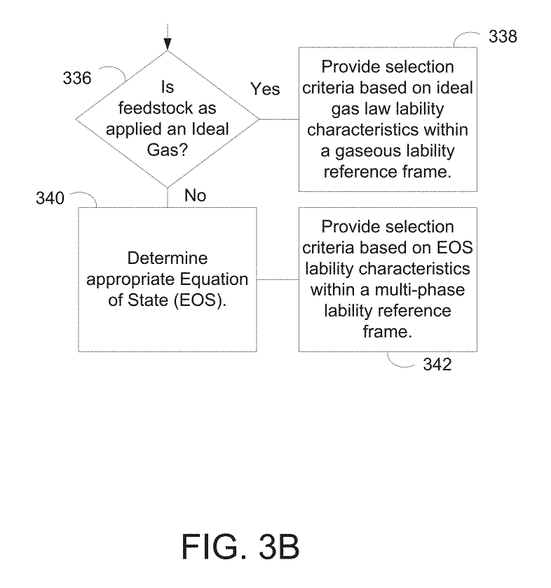

Referring now to FIG. 3 an exemplary embodiment of a method 300 according to the inventive concepts disclosed herein may include one or more of the following steps. For example, the method 300 may be a method spray and emission for food applications based on lability characteristics.

A step 302 may include determining whether the intended spray application is a food application and proceeding with spray and emission based on the determination. For example, if the spray application is not a food application, then the desired spray application and emission may be better suited to receiving data from a different database than the current database being queried. For instance, a server may contain multiple databases, with a respective database being directed towards a specific categorical constraint (e.g., food application, combustion application, nano-particle application, etc.).

A Step 304 may include ending the query in the specific database if the spray application is not a food application. In other embodiments, the Step 304 may include re-directing the query to a different database other than the current database queried.

A Step 306 may include determining which food application is the intended food application. For example, the food application may be a seed treatment application. By way of another example, the food application may be an encapsulation application.

In some embodiments, the Step 306 may include determining desired feedstock form. For example, the feedstock form may include, but is not limited to, drops, mists, fibers, extrusions, and combinations thereof. In some embodiments the Step 306 may include determining desired feedstock ingredients. For example, the feedstock ingredients may include glycerol. By way of another example, the feedstock ingredients may include a biological such as lysine. In some embodiments, the Step 306 may include determining application parameters. For example, an application temperature, pressure, velocity, and/or flow rate may be determined.

A Step 308 may include determining a feedstock applied state. For example, Step 308 may include determining that the feedstock or its ingredients are best applied in a fluid state when treating seeds. A Step 310 may include directing the query to a fluid algorithm if at Step 308 it is determined that the feedstock is best applied in a fluid state.

A Step 312 may include determining whether the feedstock is best applied in a gaseous state, or a multi-phase state. A Step 314 may include directing the query to a gas or multi-phase algorithm if at Step 312 it is determined that the feedstock is best applied in a gaseous state. It is noted that all liquids may be considered as having a multi-phase state depending on application parameters such as temperature and pressure. Therefore, the Steps 308 and 312 may be determined with respect to the application parameters.

A step 316 may include ending the query if the feedstock is not best applied at a gaseous state. In some embodiments, rather than ending the query, the query, the query may be redirected to a different database.

Referring now to FIG. 3A an exemplary embodiment of a fluid algorithm 310 according to the inventive concepts disclosed herein may include one or more of the following steps.

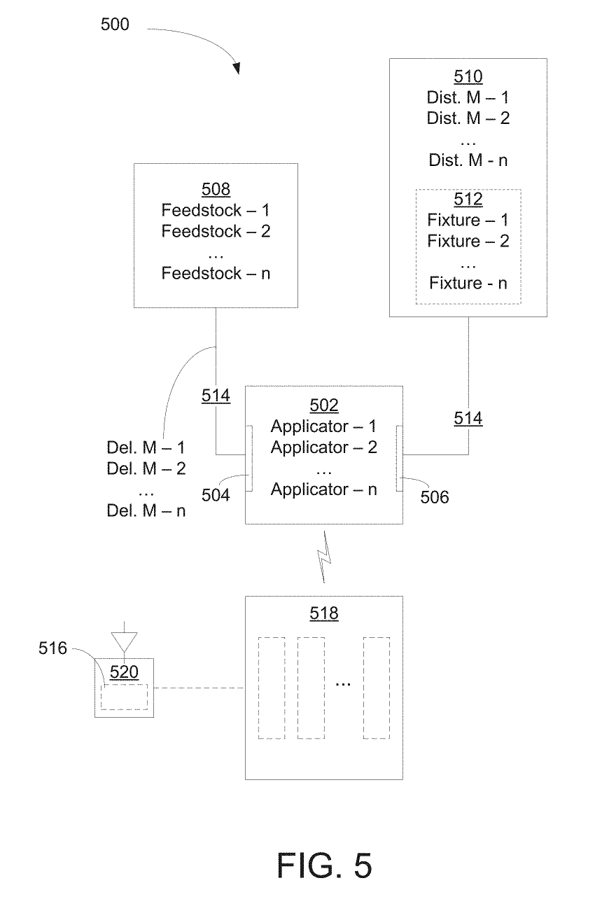

A Step 318 may include determining feedstock and propellant characteristics, individually and in combination. For example, characteristics such as viscosity, vapor pressure, moisture content, density, and combinations thereof may be determined. A Step 322 may include determining whether the feedstock as-applied is a non-Newtonian fluid. If so, then the Step 322 may include redirecting the query to a non-Newtonian fluid algorithm (e.g., FIG. 5). Otherwise, a Newtonian fluid algorithm may include determining a level of viscosity. If the viscosity is a high or medium level of viscosity, then the Step 330 may include providing spray fixture selection criteria based on lability characteristics within a viscosity lability reference frame. For instance, if the feedstock ingredient has low-level lability within a viscosity lability reference frame (e.g., the ingredient is highly viscous), then selection criteria for appropriate applicators, spray fixtures, distribution mechanisms, and/or deliver mechanisms for that level may be provided.

A Step 326 may include ending the query. In some embodiments, the Step 326 may include redirecting the query to a gaseous or multi-phase state algorithm (e.g., FIG. 3B).

A Step 334 may include providing selection criteria based on lability characteristics within a lability reference frame other than the viscosity lability reference frame. For example, the selection criteria may be based on a combinational lability reference frame such as a pH, temperature, and pressure lability reference frame. For instance, lysine may be best applied at a pH of 6-7, at a temperature of 20° C., and atmospheric pressure (e.g., 1 atm), such that the selection criteria for a spray fixture or a applicator configuration may be provided with respect to the combinational lability reference frame.

Referring now to FIG. 3B an exemplary embodiment of a gaseous state or multi-phase state algorithm 314 according to the inventive concepts disclosed herein may include one or more of the following steps.

A Step 336 may include providing selection criteria based on Ideal Gas Law lability characteristics based on a gaseous lability reference frame. For example, a degree to which a component may be alterable, or lability, may be determined based on the Ideal Gas Law, PV=nRT.

A Step 340 may include determining an appropriate Equation of State (EOS) when the Step 336 determines that the Ideal Gas Law does not apply. The appropriate EOS may depend on feedstock/propellant characteristics determined in Step 318. The appropriate EOS may include, but is not limited to, a cubic EOS (e.g., Van der Waal's EOS), a Redlich-Kwong EOS, a Soave Relich-Kwong (SRK) EOS, a Peng-Robinson EOS, a modified EOS (e.g., Van der Waal's EOS modified by Thiele's hard sphere expression), a hard chain EOS, a crossover EOS, and combinations thereof.

A Step 342 may include providing selection criteria based on the EOS lability characteristics as determined within the EOS lability reference frame.

Referring now to FIG. 5 an exemplary embodiment of a non-Newtonian fluid algorithm 322 according to the inventive concepts disclosed herein may include one or more of the following steps.

A Step 344 may include determining whether the non-Newtonian fluid is a known non-Newtonian fluid, or has any known parameters associated with it in a database. For example, the known parameters may include, but are not limited to, a “K” or an “n” parameter from the power law equation (i.e., Ostwald-de Waele equation), a Bingham number, a Hedstrom number, or combinations thereof.

A Step 346 may include ending the query if the non-Newtonian fluid is not known. In some embodiments, the Step 346 may include directing the query to another database and/or another set of logical constraints and queries. For example, the other set of logical constraints and queries may include algorithms, equations, calibrations, or experimental data steps for determining the non-Newtonian parameters needed so they might be stored in the database.

A Step 348 may include determining whether the flow can be assumed to be laminar. For example, for spray applications where the feedstock is applied using laminar non-Newtonian flow within the spray fixture and/or distribution mechanism, one or more power law lability characteristics may be used within a resistance to shear stress lability reference frame (i.e., power law may indicate a degree to which the flow is alterable, or its lability level, using power law parameters and equations).

In some embodiments, the power law lability characteristics are determined, or related, according to the following relationships:

$$τ = {K ( \frac{dV}{dy} )}^{n} \qquad ( 2 )$$

where for flow in a horizontal circular pipe, a relationship is determined as follows:

$$τ = - \frac{r}{2} \cdot ( \frac{- dP}{dx} ) \qquad ( 3 )$$

where y and r are the same from Equations (1) and (2) such that the equations may be combined and then integrated (e.g., integrate V dA=V·2πr dr) to produce the following relationship:

$$Q = \frac{n π {D}^{3}}{8 ( 3 n + 1 )} {( \frac{D}{4 K} \cdot \frac{- dP}{dx} )}^{1 / n} \qquad ( 4 )$$

Using a relationship (e.g., Equation (4)) of lability characteristics (e.g., shear stress in Equations (2) and (3)), within a shear stress lability reference frame, selection criteria may be determined. For instance, Equation (4) may be used to determine a pressure gradient (e.g., −dP/dx) for an average velocity in a pipe having a given diameter. It is noted that the above relationship assumes the spray fixture or the delivery mechanism may be characterized as a pipe. It is further noted, that the relationship may be similar, except with different friction numbers, when the fixture or the delivery mechanism is tubing, and that those skilled in the art will recognize that different types of materials and different associated friction factors are encompassed herein.

Referring now to FIG. 5, a system 500 for spray or emission for food applications based on lability characteristics is depicted. In embodiments, the system includes an applicator 502 having an adjustable speed. For example, the applicator 502 may include a tractor, a planter, a conveyor belt, a spray dryer, a continuous coating machine, a rotating drum, a spray fixture, or combinations thereof.

In embodiments, the applicator 502 has a first port 504 and a second port 506, where each of the first port 504 and the second port 506 are configured to interchangeably receive one or more components based on lability characteristics of a feedstock. For example, if lability characteristics indicate that the fluid is a highly viscous fluid, then a first one or more components may be interchanged or exchanged with a second one or more components capable of delivering and distributing the highly viscous fluid in a cost-effective manner (e.g., at a low pressure).

In embodiments, the system 500 is configured to utilize one hopper 508 of multiple possible feedstock hoppers based on the lability characteristics of the feedstock. For example, a first selected feedstock hopper 508 may have an open configuration with baffles in order to aid in quick mixing of a feedstock with multiple ingredients (e.g., seed treatment, microbes, carrier, etc.), while a second selected feedstock hopper may be a closed hopper with separate tanks for immiscible or highly labile ingredients. Thus, each of the multiple feed hoppers may have a port (e.g., third port) that is threaded to interface with a port of the applicator (e.g., first port). It is noted that while threading is used as the interfacing mechanisms, other interfacing mechanisms will be recognized by those skilled in the art, and are intended to be encompassed herein. For example, a second interfacing mechanism may include, but is not limited to, a quick release mechanism, a compressible ball-joint assembly, a Swagelok fitting, a gasket assembly, and combinations thereof.

In embodiments, the system 500 is configured to utilize one or more distribution mechanisms 510 of multiple possible distribution mechanisms. For example, a selected distribution mechanism 510 may have a port (e.g., fourth port) configured to interface with a port (e.g., second port) of the applicator. For instance, the distribution mechanism may use threading or another interfacing mechanism to interchangeably interface with a port of the applicator.

In embodiments, the system 500 is configured to utilize one or more spray fixtures 512 of multiple possible spray fixtures. For example, a selected spray fixture 512 may be one of an atomizing spray fixture, a spray fixture having needle-like columns, a spray fixture having an annular shape or resembling an annulus, a spray fixture with a 360 degree spraying radius, and combinations thereof. It is noted that examples of these and other spray fixtures are described generally in U.S. Pat. No. 9,148,994, issued on Oct. 06, 2015, filed Nov. 12, 2012, by John Alvin Eastin, et al., titled SYSTEMS FOR THE CONTROL AND USE OF FLUIDS AND PARTICLES, which is incorporated herein by reference in its entirety.

In embodiments, the distribution mechanism 510 is coupled with the feedstock hopper 508 via one or more delivery mechanisms 514 and configured to spray or emit the feedstock at least proportionally to the adjustable speed of the applicator and according to the lability characteristics of the feedstock. For example, if the applicator is a tractor, the tractor may have a PTO connection such that the speed of spray or emission from the selected distribution mechanism 510 is proportional to the speed of the tractor.

In embodiments, the system 500 includes a processor and a memory. For example, the applicator 502 may be a component of a controllable area network (CAN) having a bus, a memory, a processor. In some embodiments, the memory and the processor are configured with logical instructions (e.g., embedded or programmed into the memory) to perform the acts or steps of the methods disclosed herein.

In embodiments, the system 500 has a display 516 for displaying results of the methods disclosed herein. For example, the applicator 502 may include a vehicle with a display 516 and a user interface. The display 516 may display one or more queries, a checklist, and/or instructions for determining which applicator, spray fixture, and/or application configuration should be used for a specific food application. For instance, the display 516 may display 1) the selected feedstock hopper 508 and the selected distribution mechanism 510 based on the limited spectrum of selection criteria or configuration criteria, and 2) at least one of: the adjustment, the calibration, the dimensional, and the operational parameter. In some embodiments, displaying the selected distribution mechanism 510 include displaying a selected spray fixture 512 of multiple possible spray fixtures.

In embodiments, the display 516 is configured to display operational and assembly instructions. In some embodiments, the display may be attached to a spray vehicle for displaying spray or emission parameters to an operator of the vehicle.

In embodiments, the display 516 is communicatively coupled with a server 518 or other memory storage device. The server 518 may include multiple databases. In some embodiments, the multiple databases may be separately configured for intended spray or emission applications. In other embodiments, the multiple databases may be separately configured for intended food applications and selection criteria and configuration criteria for spray fixtures and applicators for the intended food application.

In embodiments, the display 516 may be a component of a mobile device 520 (e.g., smart phone, tablet, laptop computer, etc.) with a transceiver configured for communicating with the server 518. In this regard, the selection of a hopper 508, a selection of a distribution mechanism 510, a selection of a fixture 512, and/or a selection of an applicator 601 or an applicator configuration may be performed from the mobile device 520.

### Example 1

Flow rates (e.g., 0.14 to 5.2 liters/s) for delivering a coating to corn seed in a food application (e.g., processing, preserving, and flavor enhancement), as discussed above, were assumed to produce a desired coating thicknesses (e.g., 0.16 to 0.64 cm or 1/16^{th} to ¼ in), where glycerol was used as the coating with a density of 1.2613 g/cm³. Accordingly, one or more configuration criteria and/or selection criteria were determined based on a density lability reference frame where lability and density are inversely related (e.g., the more dense the fluid, the less labile—or subject to alteration, it is). It is noted that one or more lability characteristics (e.g., velocity and/or pressure losses) may be found based on a relationship derived from Bernouli's Equation (e.g., Equation (11)).

As shown below, Table 1 shows lability characteristics (e.g., pressure change, dP, and velocity, V1 and V2) for given pipe dimensions and the flow rates (e.g., 0.14 l/s and 5.2 l/s), which are converted to units of m³/s to make calculations easier.

TABLE 1

| | | | Flow1 (m3/s) | Flow2 (m3/s) | | |
|---|---|---|---|---|---|---|
| | | | 1.400E−04 | 5.199E−03 | | |
| | | | V1 (m/s) - | V2 (m/s) - | dP (psi) | dP (psi) |
| — | A (m) | D (m) | based on F1 | based on F2 | (V1) | (V2) |
| ⅛ in pipe | 3.716E−05 | 0.007 | 3.767 | 139.902 | 0.112 | 154.313 |
| ¼ in pipe | 6.689E−05 | 0.009 | 2.092 | 77.721 | 0.026 | 35.195 |
| ⅜ in pipe | 1.236E−04 | 0.013 | 1.132 | 42.061 | 0.006 | 7.611 |
| ½ in pipe | 1.960E−04 | 0.016 | 0.714 | 26.524 | 0.002 | 2.399 |

It is noted that at a point of contact of the propellant and feedstock (e.g., point of constructive combination) the flow rate of the feedstock may be negligible as compared to the flow rate of the propellant. Therefore, the velocities found in Table 1 above for obtaining the desired thicknesses of feedstock applied to the edible corn seed are used in Bernouli's Equation with the properties of the propellant (e.g., air at 15° C. and atmospheric pressure).

TABLE 2

| | | | Flow1 (m3/s) | Flow2 (m3/s) | | |
|---|---|---|---|---|---|---|
| | | | 1.400E−04 | 5.199E−03 | | |
| | | | V1 (m/s) - | V2 (m/s) - | dP (psi) | dP (psi) |
| — | A (m) | D (m) | based on F1 | based on F2 | (V1) | (V2) |
| ⅛ in pipe | 3.716E−05 | 0.007 | 3.767 | 139.902 | 1.086E−04 | 0.150 |
| ¼ in pipe | 6.689E−05 | 0.009 | 2.092 | 77.721 | 2.478E−05 | 0.034 |
| ⅜ in pipe | 1.236E−04 | 0.013 | 1.132 | 42.061 | 5.358E−06 | 0.007 |
| ½ in pipe | 1.960E−04 | 0.016 | 0.714 | 26.524 | 1.689E−06 | 0.002 |

The pressure drops are provided in Table 1 and Table 2 in psi for every meter of pipe because the pressure of the propellant (e.g., air supply) is known to be from 1/16^{th} psi to 10 psi or from 1/20^{th} psi to 4 psi. In this regard, because the propellant is providing the majority of the emissive (e.g., spraying) force, the pipe for the propellant should not be so long such that the pressure of the propellant is significantly reduced prior to the point of contact with the feedstock. However, as inferred from Table 2, the pipe delivering the air may be very long before significant pressure loss occurs.

However, the same is not true of the feedstock. Due to the greater density of fluid being moved, greater pressure losses occur in the pipes carrying the feedstock (e.g., see Table 1). Based on the pressure losses at the given diameters within the density lability reference frame, upper threshold ratios for each type of pipe may be determined. The determined ratios are a ratio of length to diameter, where the length is a maximum length of pipe that could follow the point of contact (e.g., point of constructive combination), and the spray fixture configuration design must have ratios less than the determined threshold ratios in order to ensure that the pressure loss is not so great that there is no emission (e.g., spraying) occurring at the outlet of the nozzle. The determined ratios are provided in Table 3 below based on a pressure of 10 psi and the desired flow rate of 5.2 liters per second (e.g., as provided by propellant).

TABLE 3

| | L/D |
|---|---|
| Pipe type | ratio |
| ⅛ in pipe | N/A |
| ¼ in pipe | 10 |
| ⅜ in pipe | 85 |
| ½ in pipe | 243 |

It is noted that the “N/A” for the ⅛ in (0.3175 cm) pipe means that extremely little or no pipe should follow the point of contact. In other words, the propellant should be angled such that it contacts the feedstock flowing out of a ⅛ in pipe directly, with negligible lengths of pipe restricting the flow of the feedstock after the contact. For example, in this case the air could act as a seed knife, separating the feedstock from the delivery tube at the point of constructive contact.

On the other end of the spectrum, at the desired flow rate of 0.14 liters per second and a pressure of 1/20^{th} psi, the determined length to diameter design ratios for length of pipe following the point of contact are provided in Table 4 below.

TABLE 4

| | L/D |
|---|---|
| Pipe type | ratio |
| ⅛ in pipe | 45 |
| ¼ in pipe | 191 |
| ⅜ in pipe | 705 |
| ½ in pipe | 1802 |

An inference that can be made based on Table 3 and Table 4, is that if higher volumetric emission (e.g., spray) is desired for a low lability fluid (e.g., glycerol) within the density lability reference frame, then less pipe (or tubing) should follow the point of constructive contact. In some embodiments, the distribution mechanism (e.g., nozzle) uses a very short length of tubing following the point of contact, which means that the pressure loss from the flow of the high viscosity fluid (e.g., glycerol) will be minimal. In this regard, the distribution mechanism may include an adjustable contact plate, which may adjust a size of an opening through which the feedstock-propellant mixture flows, thereby adjusting the emission, expulsion, or spraying rate and pattern.

In embodiments, a length of pipe following the point of contact of the propellant and feedstock is from 0.5 to 1 to 2 inch (0.0127 to 0.0254 m). The pipe used for the feedstock is ½ inch, schedule 40 pipe, and the pipe used for the propellant is ¼ inch, schedule 40 pipe of a length of 5 m or less. The pressure loss due to the feedstock flowing over the 1 inch length of pipe following the point of contact, and the pressure loss of air flowing over 5 m or less of pipe, is approximately 1.73 (0.5 in) to 1.76 (1 in) to 1.82 psi (2 in), so of the 10 psi provided by the air. Therefore, approximately 81.8% to 82.4% to 82.7% of the air is available to propel the 5.2 l/s of feedstock (e.g., glycerol) from the nozzle with these lengths of pipe following the point of contact. In these embodiments, the propellant (e.g., air) is supplied at a pressure from 2 to 10 psi.

When the flow rate used is the desired 0.14 liters per second, then the pressure loss over the 0.5 to 1 to 2 inches of pipe following the point of contact is approximately 0.00125 (for 0.5 in) to 0.00128 (for 1 in) to 0.00132 psi (for 2 in), which is approximately 2.5% to 2.6% to 2.64% of a 1/20^{th} psi air supply. In other words, 97.36% to 97.4% to 97.5% of the propellant (e.g., air supplied at 1/20^{th} psi) is available to propel the 0.14 l/s of feedstock.

It is noted that if, schedule 40 steel pipe is assumed, then a surface roughness value, E, is 0.0018 in. In some embodiments, a drawn tubing (e.g., brass, lead, glass, etc.) is used, which has a surface roughness value of 0.00006 in. It is further noted that an equivalent length of approximately 20 is used, or a resistance coefficient of approximately 0.4, for two angles that may exist in embodiments of a nozzle disclosed herein (e.g., two angles are approximated as standard tee). It is further noted that these numbers may increase if the amount of pressure that a feedstock contributes to expel the propellant-feedstock mixture from a nozzle is increased.

It is to be understood that embodiments of the methods according to the inventive concepts disclosed herein may include one or more of the steps described herein. Further, such steps may be carried out in any desired order and two or more of the steps may be carried out simultaneously with one another. Two or more of the steps disclosed herein may be combined in a single step, and in some embodiments, one or more of the steps may be carried out as two or more sub-steps. Further, other steps or sub-steps may be carried in addition to, or as substitutes to one or more of the steps disclosed herein.

From the above description, it is clear that the inventive concepts disclosed herein are well adapted to carry out the objects and to attain the advantages mentioned herein as well as those inherent in the inventive concepts disclosed herein. While presently preferred embodiments of the inventive concepts disclosed herein have been described for purposes of this disclosure, it will be understood that numerous changes may be made which will readily suggest themselves to those skilled in the art and which are accomplished within the broad scope and coverage of the inventive concepts disclosed and claimed herein.

1. A method for labile-based spray and emission for food applications, comprising:
determining a lability reference frame for a feedstock;
comparing first lability characteristics of the feedstock at a first point in the lability reference frame with second lability characteristics of the feedstock at a second point in the lability reference frame to determine a level of lability of the feedstock;
limiting a spectrum of selection criteria or configuration criteria based on the level of lability; and
converging on a value to determine an adjustment, a calibration, a dimensional, or an operational parameter using the limit and at least one of the first lability characteristics and the second lability characteristics.

2. The method of claim 1, wherein limiting a spectrum of selection criteria or configuration criteria based on the level of lability comprises limiting a selection criteria for selecting at least one of a spray fixture, an applicator, and an applicator configuration for a food application, and wherein the food application comprises at least one of: seed treatment, planting, pest control, plant health, harvest, desiccation, processing, preservation, flavor enhancement, encapsulation, suspension, emulsion, packaging, shipping, and storage application.

3. (canceled)

4. The method of claim 1, wherein converging on a value to determine an adjustment, a calibration, a dimensional, or an operational parameter using the limit and at least one of the first lability characteristics and the second lability characteristics comprises converging on an opening dimension for a nozzle of a spray fixture.

5. The method of claim 4, wherein the opening is an opening resembling at least one of: an annulus, an annulus segment, a space between adjustable contact plates, a space between adjustable opposing plates, and a needle-like column.

6. The method of claim 1, wherein converging on a value to determine an adjustment, a calibration, a dimensional, or an operational parameter using the limit and at least one of the first lability characteristics and the second lability characteristics comprises determining an auger speed.

7. (canceled)

8. A method of spray or emission for food applications based on lability characteristics, comprising:
selecting one or more spray or emission applications from a plurality of food applications;
determining one or more spray or emission forms and associated spray or emission parameters;
determining feedstock characteristics including at least one of lability, thermodynamic, fluidic, and kinematic properties and associating the feedstock characteristics with a lability reference frame; and
providing spray fixture selection or configuration criteria based on the lability reference frame, the feedstock characteristics, the emission form, and the one or more spray or emission forms.

9. The method of claim 8, wherein the plurality of food applications comprises a seed treatment application, a planting application, a pest control application, a plant health application, a harvest application, a desiccation application, a processing application, a preservation application, a flavor enhancement application, an encapsulation application, a suspension application, an emulsion application, a packaging application, a shipping application, and a storage application.

10. The method of claim 8, wherein the one or more spray or emission forms comprises at least one of: drops, globules, fibers, mists, and extrusions.

11. The method of claim 8, wherein the associated spray or emission parameters comprise at least one of: a flow rate, a coating thickness, a coating volume, an application temperature, an application pressure, and an environmental condition.

12. The method of claim 8, wherein determining feedstock characteristics including at least one of lability, thermodynamic, fluidic, and kinematic properties and associating the feedstock characteristics with a lability reference frame comprises determining a dynamic, plastic, or rheological viscosity.

13. The method of claim 12, wherein providing spray fixture selection or configuration criteria based on the lability reference frame, the feedstock characteristics, the emission form, and the selected spray or emission form comprises providing a list of possible spray fixtures or possible applicator configurations that are capable of spraying or emitting the feedstock having the dynamic, plastic, or rheological viscosity.

14. A system for spray or emission for food applications based on lability characteristics, comprising:
an applicator having an adjustable speed and comprising a first port, a second port, and at least one adjustable valve, each of the first port and the second port being configured for interchangeably receiving one or more components based on lability characteristics of a feedstock;
a selected feedstock hopper having a third port configured to interface with the first port; and
a selected distribution mechanism having a fourth port configured to interface with the second port, the distribution mechanism being coupled with the feedstock hopper via one or more delivery mechanisms and configured to spray or emit the feedstock at least proportionally to the adjustable speed and according to the lability characteristics of the feedstock, the spray or emission being adjustable using the adjustable valve.

15. The system of claim 14, wherein the applicator comprises at least one of: a tractor, a planter, a conveyor belt, a spray dryer, a continuous coating machine, a rotating drum, and a spray fixture.

16. The system of claim 14, wherein the first port and the second port of the applicator have a threading for interchangeably receiving the one or more components, and wherein the one or more components comprise the selected feed hopper and the selected distribution mechanism.

17. The system of claim 16, wherein the threading is a first threading, the third port having a second threading for operatively interfacing with the first port.

18. The system of claim 17, wherein the fourth port has a fourth threading for operatively interfacing with the first port.

19. The system of claim 14, wherein the applicator comprises a memory and a processor, the memory and the processor configured to perform the acts of:
determining a lability reference frame for the feedstock;
comparing first lability characteristics of the feedstock at a first point in the lability reference frame with second lability characteristics of the feedstock at a second point in the lability reference frame to determine a level of lability of the feedstock;
limiting a spectrum of selection criteria or configuration criteria based on the level of lability; and
converging on a value to determine an adjustment, a calibration, a dimensional, or an operational parameter using the limit and at least one of the first lability characteristics and the second lability characteristics.

20. The system of claim 19, wherein the applicator comprises a display configured for displaying 1) the selected feedstock hopper and the selected distribution mechanism based on the limited spectrum of selection criteria or configuration criteria, and 2) at least one of: the adjustment, the calibration, the dimensional, and the operational parameter.

21. The method of claim 6, wherein the first lability characteristics or the second lability characteristics include at least one of pressure and one or more sensitivity or damageable characteristics of a feedstock ingredient, and wherein the auger speed and an auger thread design are determined based on the at least one of pressure and one or more sensitivity or damageable characteristics of the feedstock ingredient.

22. The method of claim 1, wherein limiting a spectrum of selection criteria or configuration criteria based on the level of lability comprises a determination that a spray fixture uses a propellant, and wherein the propellant used by the spray fixture is selected based on at least one of the first lability characteristics and the second lability characteristics of the feedstock.
